(19) United States
(12) Patent Application Publication
(10) Pub. No.: US 2024/0408107 A1
(43) Pub. Date: Dec. 12, 2024

(54) LACTAM PYRROLIDINE-PYRAZOLES AS PYRUVATE KINASE ACTIVATORS

(71) Applicant: Global Blood Therapeutics, Inc., South San Francisco, CA (US)

(72) Inventors: Shahul Nilar, Foster City, CA (US); Lina Q. Setti, Fremont, CA (US); Chul Yu, San Ramon, CA (US); Ming Yu, Foster City, CA (US)

(73) Assignee: Global Blood Therapeutics, Inc., South San Francisco, CA (US)

(21) Appl. No.: 18/698,516

(22) PCT Filed: Oct. 5, 2022
(86) PCT No.: PCT/US2022/077619

§ 371 Date: Apr. 4, 2024

Related U.S. Application Data
(60) Provisional application No. 63/252,947, filed on Oct. 6, 2021.

(51) Int. Cl. A61K 31/553; A61K 31/4162; A61K 31/428; A61K 31/4725; C07D 487/04

(52) U.S. Cl. CPC A61K 31/553; A61K 31/4162; A61K 31/428; A61K 31/4725; C07D 487/04

(57) ABSTRACT

The subject matter described herein is directed to pyruvate kinase activating compounds of Formula I and pharmaceutical salts thereof, methods of preparing the compounds, pharmaceutical compositions comprising the compounds and methods of administering the compounds for the treatment of diseases associated with PKR and/or PKM2, such as pyruvate kinase deficiency, sickle cell disease, and beta-thalassemia.

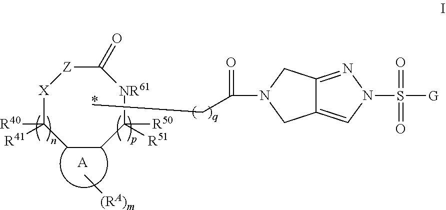

### CROSS REFERENCE TO RELATED APPLICATIONS

This application claims the benefit of priority to U.S. Provisional Patent Application No. 63/252,947, filed on Oct. 6, 2021, the contents of which is incorporated by reference herein in their entirety for all purposes.

### FIELD

The subject matter described herein is directed to pyruvate kinase activating compounds, methods of making the compounds, pharmaceutical compositions, and their use in the treatment of diseases associated with PKR and/or PKM2.

### BACKGROUND

Pyruvate kinase (PK) is an essential component of cellular metabolism, converting ADP and phosphoenolpyruvate (PEP) to pyruvate in the final step of glycolysis. There are four unique isoforms of pyruvate kinase that vary in concentration by different tissue types (Dayton et al., EMBO Rep. 2016 17(12):1721-1730); Israelsen and Vander Heiden, Semin. Cell Dev. Biol. 2015 July; 43:43-51; Mazurek, Int. J. Biochem. Cell Biol. 2011 July; 43(7):969-80). Each isomer is responsible for catalyzing the production of pyruvate and ATP, while being regulated in a manner respective to each tissue type.

Pyruvate kinase in the liver (PKL) and pyruvate kinase from erythrocytes/red blood cells (PKR) are tetrameric enzymes that depend on an endogenous activator called fructose-1,6-bisphosphate (FBP) for activation (Koler and Vanbellinghen, Adv. Enzyme Regul. 1968 6:127-42; Taylor and Bailey, Biochem J. 1967 967 February; 102(2):32C-33C). The PKM1 isoform is found in the brain, heart, and skeletal muscle where it functions as a stable and constitutively active tetrameric protein. PKM1 therefore does not require FBP for activation. The PKM2 isomer is expressed in most tissue types, including cancers, developing embryos, and all proliferative tissues. Similar to PKR and PKL, PKM2 requires FBP for allosteric activation via stabilization of the enzyme in a tetrameric and most active form (Cardenas and Dyson, J. Exp. Zool. 1978 June; 204(3):361-7; Imamura and Tanaka, J. Biochem. 1972 June; 71(6):1043-51; Strandholm et al., Arch. Biochem. Biophys. 1976 March; 173(1):125-31).

Mature red blood cells (RBCs) rely on glycolysis for energy production. All tumor cells exclusively express the PKM2 isoform, suggesting that PKM2 would be a good target for cancer therapy. PKM2 is also expressed in adipose tissue and activated T-cells. Thus, controlling the regulation of PKM2 activity may be effective for treatment of obesity and diabetes in addition to cancer.

Genetic mutations in all glycolytic enzymes result in hemolytic anemia (Van Wijk and Van Solinge, Blood 2005 106 (13): 4034-4042.). Mutations in PKL and PKR that result in a loss of function are known to cause PK deficiency (PKD) and the clinical manifestation of these mutations appear confined to RBCs.

There are greater than 200 known and reported mutations associated with PKD reported worldwide (Zanella et al., J. Haematol. 2005 July; 130(1):11-25). Some mutations directly disrupt catalytic activity of the PK enzyme while other mutations disrupt the interactions between monomers that stabilize the active tetrameric enzyme. The mutation of Arginine residue 510 to Glutamine is one of the most common mutations found in North American and European patients, ˜40% of patients, and is known to disrupt stability of the PKR tetramer (Kedar et al., Clin. Genet. 2009 February; 75(2):157-62; Wang et al., Blood 2001 Nov. 15; 98(10):3113-20).

Patients with PKD suffer from chronic hemolytic anemia in addition to multiple co-morbidities. Blood transfusions and splenectomy are common treatments and it has been suggested that gene therapies could be used for treatment of PKD in the near future (Garcia-Gomez et al., Molecular Therapy 2016 Aug. 1; 24(7); Grace et al., Am. J. Hematol. 2015 September; 90(9):825-30).

The number of PKD patients worldwide is unknown; however, the prevalence in the general Caucasian population is estimated to be around 1:20,000 people with 51 cases per million people in North America (Beutler and Gelbart, Blood 2000 Jun. 1; 95(11):3585-8).

There are no approved drugs for the treatment of PKD. Clinically, hereditary PKR deficiency disorder manifests as a non-spherocytic hemolytic anemia. The clinical severity of this disorder ranges from no observable symptoms in fully-compensated hemolysis to potentially fatal severe anemia requiring chronic transfusions and/or splenectomy at early development or during physiological stress. For some of the most severe cases, while extremely rare population-wise with estimated frequency of 1 in 20,000 patients, there is no disease modifying treatment besides transfusions. These hereditary non-spherocytic hemolytic anemia patients present a clear unmet medical need. RBCs from patients with either sickle cell anemia or with beta-thalassemia suffer from increased ATP demand to maintain overall RBC health. The activation of PKR in both sickle cell disease patients and beta-thalassemia patients could lead to improved cell fitness and survival.

What is therefore needed and not effectively addressed by the art are compounds that act as pyruvate kinase activators that have desired efficacy and therapeutic potential. This problem as well as others stemming from pyruvate kinase deficiency are addressed by the subject matter described herein.

### BRIEF SUMMARY

In certain embodiments, the subject matter described herein is directed to compounds of Formula I, which include compounds of Formulae Ia, Ib, Ib2, Ib3, Ic, Id, Id2, Id3, II and III, and pharmaceutically acceptable salts thereof and uses of the compounds as described herein. In certain embodiments, the subject matter described herein is directed to a compound of Formula I, Ia, Ib, Ib2, Ib3, Ic, Id, Id2, Id3, II, or III, or a pharmaceutically acceptable salt thereof.

In certain embodiments, the subject matter described herein is directed to a pharmaceutical composition comprising a compound of Formula I, Ia, Ib, Ib2, Ib3, Ic, Id, Id2, Id3, II, or III, or a pharmaceutically acceptable salt thereof.

In certain embodiments, the subject matter described herein is directed to a method of treating a disease or disorder associated with modulation of pyruvate kinase (PKR) and/or PKM2 in a subject, comprising administering to the subject an effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I, Ia, Ib, Ib2, Ib3, Ic, Id, Id2, Id3, II, or III.

In certain embodiments, the subject matter described herein is directed to a method of activating PKR and/or PKM2 in a subject, comprising administering to the subject an effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I.

In certain embodiments, the subject matter described herein is directed to a method of treating a subject afflicted with a disease associated with decreased activity of PKR and/or PKM2, comprising administering to the subject an effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I, Ia, Ib, Ib2, Ib3, Ic, Id, Id2, Id3, II, or III.

Other embodiments are also described.

### DETAILED DESCRIPTION

Described herein are pyruvate kinase activators of Formula I, Ia, Ib, Ib2, Ib3, Ic, Id, Id2, Id3, II, or III, methods of making the compounds, pharmaceutical compositions comprising the compounds, and their use in the treatment of diseases associated with decreased pyruvate kinase activity.

PKR activating compounds could be used to treat patients with beta-thalassemia and sickle cell anemia (Alli et al., Hematology 2008 December; 13(6):369-72; Kung et al., Blood 2017 Sep. 14; 130(11):1347-135). As shown in a mouse model of beta-thalassemia, the PKR activator in clinical trials, AG-348, increased PK activity and ATP levels, as well as improved RBC parameters. Similar results were obtained from treating human beta-thalassemia RBCs ex vivo (Kuo et al., Mitapivat (AG-348), an oral PK-R activator, in adults with non-transfusion-dependent thalassemia: A phase 2, open-label, multicenter study in progress; 61^{st} Am. Soc. Hematol. Annual Meeting, December 2019).

The compounds of Formula I, Ia, Ib, Ib2, Ib3, Ic, Id, Id2, Id3, II, or III described herein are useful in the treatment of diseases or disorders associated with pyruvate kinase function. As demonstrated by the biochemical assays described herein, the compounds of Formula I, Ia, Ib, Ib2, Ib3, Ic, Id, Id2, Id3, II, or III activate PKR and/or PKM2. In certain embodiments, the compounds described herein are more effective at activating PKR and/or PKM2 than AG-348. The compounds of Formula I, Ia, Ib, Ib2, Ib3, Ic, Id, Id2, Id3, II, or III are useful in the treatment of diseases including, but not limited to, pyruvate kinase deficiency and sickle cell disease, such as sickle cell anemia, and beta-thalassemia. Also, the compounds are methods described herein are useful in treating cancer.

Pyruvate kinase activators are needed that also possess additional beneficial properties such as improved solubility, stability, and/or potency. An advantage of the pyruvate kinase activator compounds of Formula I, Ia, Ib, Ib2, Ib3, Ic, Id, Id2, Id3, II, or III described herein is their preparation in sufficient yields by the synthetic routes disclosed herein.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±50%. In certain other embodiments, the term “about” includes the indicated amount ±20%. In certain other embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

The term “alkylene” by itself or as part of another substituent means a divalent radical derived from an alkane, such as, methylene —CH₂—, ethylene —CH₂CH₂—, and the like. As an example, a “hydroxy-methylene” refers to HO—CH₂—*, where * is the attachment point to the molecule.

Unless indicated explicitly otherwise, where combinations of groups are referred to herein as one moiety, e.g., arylalkyl or aralkyl, the last mentioned group contains the atom by which the moiety is attached to the rest of the molecule.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy. Additional examples are C₁-C₃ alkoxy and C₁-C₆ alkoxy, which refer to “C₁-C₃ alkyl-O” or “C₁-C₆ alkyl-O,” respectively.

“Amino” refers to the group —NR^{y}R^{z} wherein R^{y} and R^{z} are independently hydrogen, alkyl, cycloalkyl, heterocyclyl, aryl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like. Furthermore, “halo-C₁-C₃ alkyl” refers to an alkyl group of 1 to 3 carbons wherein at least one hydrogen atom is replaced by a halogen. Additionally, “halo-C₁-C₆ alkyl” refers to an alkyl group of 1 to 6 carbons wherein at least one hydrogen atom is replaced by a halogen.

“Haloalkoxy” refers to an alkoxy group as defined above, wherein one or more (e.g., 1 to 6, or 1 to 3) hydrogen atoms are replaced by a halogen. As used herein, “halo-C₁-C₃ alkoxy” refers to a C₁-C₃ alkyl-O— group wherein at least one of the hydrogen atoms of the carbon chain is replaced by a halogen.

“Hydroxyalkyl” or “hydroxyalkylene” and the like refers to an alkyl or alkylene group as defined above, wherein one or more (e.g., 1 to 6, or 1 to 3) hydrogen atoms are replaced by a hydroxy group. By way of example, the term “hydroxy-C₁-C₃ alkyl” or “hydroxy-C₁-C₃ alkylene” refers to a one to three carbon alkyl chain where one or more hydrogens on any carbon is replaced by a hydroxy group, in particular, one hydrogen on one carbon of the chain is replaced by a hydroxy group.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 6-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- or 6-ring atoms, it is also referred to herein as a 4- or 6-membered heterocyclyl. When the heterocyclyl ring contains 5- to 7-ring atoms, it is also referred to herein as a 5- to 7-membered heterocyclyl. When the heterocyclyl ring contains 5- to 10-ring atoms, it is also referred to herein as a 5- to 10-membered heterocyclyl. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkylene, alkoxy, haloalkyl, haloalkoxy, cycloalkyl, aryl, heterocyclyl, and/or heteroaryl) wherein at least one (e.g., 1 to 5, 1 to 4, or 1 to 3) hydrogen atom is replaced by a bond to a non-hydrogen atom such as, but not limited to alkyl, alkoxy, amino, aryl, aralkyl, carboxyl, carboxyl ester, cyano, cycloalkyl, halo, haloalkyl, haloalkoxy, hydroxyalkyl, heteroaryl, heterocyclyl, —NHNH₂, hydroxy, oxo, nitro, —S(O)OH, —S(O)₂OH, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, oxo, alkyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkoxy, aryl, cycloalkyl, haloalkyl, heterocyclyl, and/or heteroaryl. In certain embodiments, “substituted” also means any of the above groups in which one or more (e.g., 1 to 5, 1 to 4, or 1 to 3) hydrogen atoms are replaced by a bond to an amino, cyano, hydroxyl, nitro, oxo, halo, alkyl, alkoxy, alkylamino, aryl, cycloalkyl, haloalkyl, heterocyclyl, N-heterocyclyl, heteroaryl, or two of R^{g} and R^{h} and R are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), mixture of isomers (such as a mixture of stereoisomers), prodrug, and metabolite of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), ti(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds described herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The subject matter described herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The subject matter described herein contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The term, “metabolite,” as used herein refers to a resulting product formed when a compound disclosed herein is metabolized. As used herein, the term “metabolized” refers to the sum of processes (including but not limited to hydrolysis reactions and reactions catalyzed by enzymes) by which a particular substance, such as a compound disclosed herein, is changed by an organism. For example, an aldehyde moiety (—C(O)H) of the compounds described herein may be reduced in vivo to a —CH₂OH moiety.

As used herein, the term “activator” refers to a compound of Formula I, Ia, Ib, Ib2, Ib3, Ic, Id, Id2, Id3, II, or III, or a pharmaceutically acceptable salt thereof that increases the activity of pyruvate kinase R (PKR) or pyruvate kinase M2 (PKM2), unless specified otherwise. By “activate” herein is meant to increase the activity of PKR or PKM2 activity to a level that is greater than the basal levels of activity for PKR or PKM2 in the presence of the compound. In some embodiments, the term “activate” means an increase in the activity of PKR or PKM2 of at least about 5%, at least about 10%, at least about 20%, at least about 25%, at least about 50%, at least about 60%, at least about 70%, at least about 80%, at least about 90%, or at least about 95%. In other embodiments, activate means an increase in PKR or PKM2 activity of about 5% to about 25%, about 25% to about 50%, about 50% to about 75%, or about 75% to 100%. In some embodiments, activate means an increase in PKR or PKM2 activity of about 95% to 100%, e.g., an increase in activity of 95%, 96%, 97%, 98%, 99%, or 100%. Such increases can be measured using a variety of techniques that would be recognizable by one of skill in the art, including in vitro assays.

As used herein, the term “pyruvate kinase activator” and the like refers to a compound that activates, increases, or modulates one or more of the biological activities of pyruvate kinase. The activity could increase, for example, at least about 5%, 10%, 15%, 20%, 25%, 30%, 35%, 40%, 45%, 50%, 55%, 60%, 65%, 70%, 75%, 80%, 85%, 95% or 100% of the activity of pyruvate kinase compared to an appropriate control. The increase can be a statistically significant increase.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a pyrukate kinase deficiency (PKD). The therapeutically effective amount may vary depending on the subject, and disease or condition being treated, the weight and age of the subject, the severity of the disease or condition, and the manner of administering, which can readily be determined by one of ordinary skill in the art.

Additional definitions may also be provided below as appropriate.

### II. Compounds

In certain embodiments, the subject matter described herein is directed to compounds of Formula I, and pharmaceutically acceptable salts thereof:

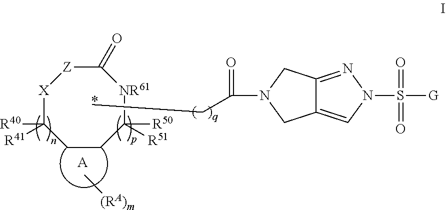

or a pharmaceutically acceptable salt thereof;
wherein,
   m is 0, 1, or 2;
   Ring A is a fused phenyl or 5- or 6-membered heteroaryl;
   R^{A}, in each instance, is independently selected from the group consisting of C₁-C₃ alkyl, C₁-C₃ alkoxy, hydroxy, halo-C₁-C₃ alkyl, halo-C₁-C₃ alkoxy, and halo;
   Z is a bond or CR²⁰R²¹;
      wherein, R²⁰ and R²¹ are each independently hydrogen or C₁-C₃ alkyl;
   X is NR³⁰, O or CR³⁰R³¹;
      wherein, R³⁰ and R³¹ are each independently hydrogen or C₁-C₃ alkyl; R⁴⁰, R⁴¹, R⁵⁰, R⁵¹ and R⁶¹ are each independently hydrogen or C₁-C₃ alkyl;
   n is 0 or 1;
   p is 0 or 1;
   * is an attachment point at any one of R²⁰, R²¹, R³⁰, R³¹, R⁴⁰, R⁴¹, R⁵⁰, R⁵¹ and R⁶¹;
   q is 0 or 1;
   G is:
      a) a 5-membered heteroaryl ring optionally substituted with one or two substituents, each independently selected from the group consisting of halo, C₁-C₃ alkyl, C₁-C₃ alkoxy, halo-C₁-C₃ alkoxy, and halo-C₁-C₃ alkyl;
   or,
      b)

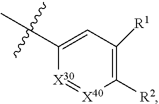

wherein,
X³⁰ and X⁴⁰ are each independently N or CH, provided that only one of X³⁰ and X⁴⁰ can be N;
R¹ and R² are each independently selected from the group consisting of hydrogen, C₁-C₃ alkyl, amino, halo, halo-C₁-C₃ alkyl, halo-C₁-C₃ alkoxy, C₁-C₃ alkoxy, hydroxy, —O—R^{aa}, —(C₁-C₃ alkoxy)-R^{aa}, and C₃-C₇ cycloalkyl;
R^{aa} is 4- to 7-membered heterocyclyl or C₃-C₇ cycloalkyl;
or,
R¹ and R², together with the carbon to which each is attached, form a 5- or 6-membered heteroaryl or 5- to 7-membered heterocyclyl containing one or two heteroatoms;
wherein said heteroaryl or heterocyclyl comprising R¹ and R² is optionally substituted with one or two substituents, each independently selected from the group consisting of C₁-C₃ alkyl, halo-C₁-C₃ alkyl, C₁-C₃ alkoxy, halo, and halo-C₁-C₃ alkoxy.

As set forth in the formulae described herein, the symbol * represents an attachment point at any one of R²⁰, R²¹, R³⁰, R³¹, R⁴⁰, R⁴¹, R⁵⁰, R⁵¹ and R⁶¹. When * is at a position, it replaces the hydrogen or alkyl previously defined as being R²⁰, R²¹, R³⁰, R³¹, R⁴⁰, R⁴¹, R⁵⁰, R⁵¹ or R⁶¹. Examples of such replacements are shown in Formulae Ib, Ic, and Id, described herein.

In certain embodiments, compounds include those of Formula I, or pharmaceutically acceptable salts thereof, where ring A is phenyl.

In certain embodiments, compounds include those of Formula Ia, or pharmaceutically acceptable salts thereof:

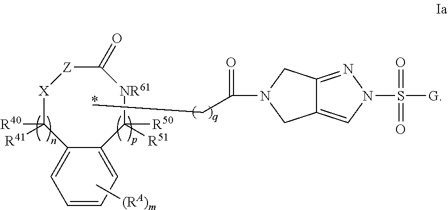

In certain embodiments, compounds include those of Formula I or Ia, or pharmaceutically acceptable salts thereof, where Z is a bond. In certain embodiments, compounds include those of Formula I or Ia, or pharmaceutically acceptable salts thereof, where Z is CR²⁰R²¹. In certain embodiments, compounds include those of Formula I or Ia, or pharmaceutically acceptable salts thereof, where R²⁰ and R²¹ are each hydrogen.

In certain embodiments, compounds include those of Formula I or Ia, or pharmaceutically acceptable salts thereof, where X is CR³⁰R³¹. In certain embodiments, compounds include those of Formula I or Ia, or pharmaceutically acceptable salts thereof, where R³⁰ is hydrogen. In certain embodiments, compounds include those of Formula I or Ia, or pharmaceutically acceptable salts thereof, where R³⁰ is methyl.

In certain embodiments, compounds include those where R³¹ is * and the compound is of Formula Ib, or pharmaceutically acceptable salts thereof:

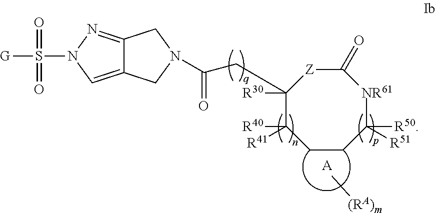

In certain embodiments, compounds include those of Formula I, or Ia, or pharmaceutically acceptable salts thereof, where R³¹ is hydrogen.

In certain embodiments, compounds include those of Formula I, Ia, or Ib, or pharmaceutically acceptable salts thereof, where X is O. In certain embodiments, compounds include those of Formula I, Ia, or Ib, or pharmaceutically acceptable salts thereof, where X is NR³⁰. In certain embodiments, compounds include those of of Formula I, Ia, or Ib, or pharmaceutically acceptable salts thereof, where R³⁰ is hydrogen. In certain embodiments, compounds include those of of Formula I, Ia, or Ib, or pharmaceutically acceptable salts thereof, where R⁶¹ is hydrogen.

In certain embodiments, compounds include those of Formula Ic, or pharmaceutically acceptable salts thereof, where R⁶¹ is *

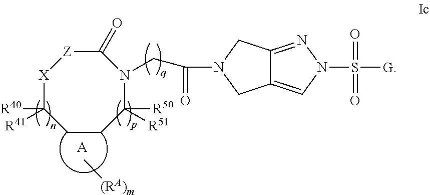

In certain embodiments, compounds include those of Formula I, Ia, Ib, or Ic, or pharmaceutically acceptable salts thereof, where n is 0.

In certain embodiments, compounds include those where p is 1, R⁵⁰ is hydrogen and R⁵¹ is * and the compound is of Formula Id, or pharmaceutically acceptable salts thereof:

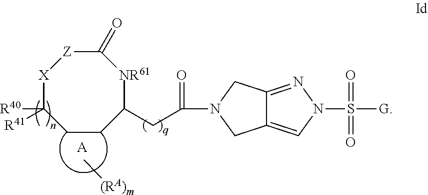

In certain embodiments, compounds include those of Formula I, Ia, Ib, or Ic, or pharmaceutically acceptable salts thereof, where p is 0.

In certain embodiments, compounds include those where R³¹ is * and the compound is of Formula Ib, or pharmaceutically acceptable salts thereof:

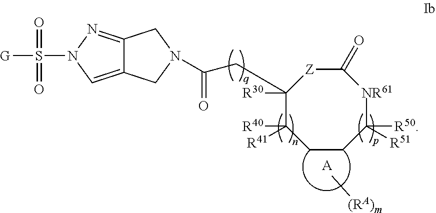

where Z is a bond; R³⁰ is hydrogen or methyl; R⁶¹ is hydrogen; n is 0; and p is 0.

In certain embodiments, compounds include those where R⁶¹ is * and the compound is of Formula Ic, or pharmaceutically acceptable salts thereof:

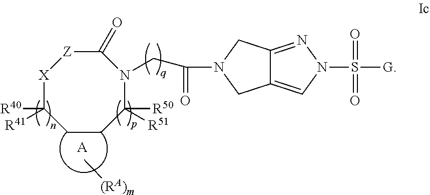

where Z is a bond; X is O, NH, or CH₂; n is 0; and p is 0.

In certain embodiments, compounds include those where p is 1, R⁵⁰ is hydrogen, R⁵¹ is * and the compound is of Formula Id, or pharmaceutically acceptable salts thereof:

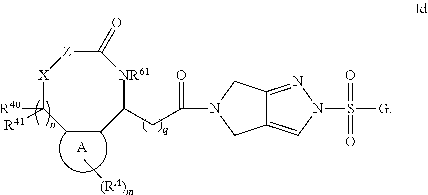

where Z is CH₂; X is O; n is 0; and R⁶¹ is hydrogen.

In certain embodiments, compounds include those where p is 1, R⁵⁰ is hydrogen, R⁵¹ is * and the compound is of Formula Id, or pharmaceutically acceptable salts thereof:

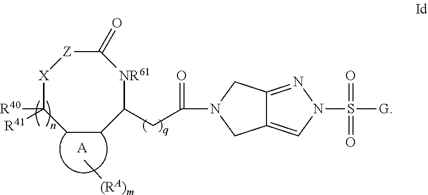

where Z is a bond; X is CH₂; n is 0; and, R⁶¹ is hydrogen.

In certain embodiments, compounds include those of Formula I, Ia, Ib, Ic, or Id, or pharmaceutically acceptable salts thereof, where q is 0. In certain embodiments, compounds include those of Formula I, Ia, Ib, Ic, or Id, or pharmaceutically acceptable salts thereof, where q is 1.

In certain embodiments, compounds include those of Formula I, Ia, Ib, Ic, or Id, or pharmaceutically acceptable salts thereof, where G is phenyl, wherein R¹ is hydrogen and R² is halo-C₁-C₃ alkoxy. In certain embodiments, compounds include those of Formula I, Ia, Ib, Ic, or Id, or pharmaceutically acceptable salts thereof, where R² is —OCH₂F, —OCHF₂, or —OCF₃. In certain embodiments, compounds include those of Formula I, Ia, Ib, Ic, or Id, or pharmaceutically acceptable salts thereof, where R² is —OCHF₂. In certain embodiments, compounds include those of Formula I, Ia, Ib, Ic, or Id, or pharmaceutically acceptable salts thereof, where G is phenyl, wherein R¹ and R², together with the carbon to which each is attached, form a 5-membered heteroaryl. In certain embodiments, compounds include those of Formula I, Ia, Ib, Ic, or Id, or pharmaceutically acceptable salts thereof, where R¹ and R², together with the carbon to which each is attached, form a thiazolyl. In certain embodiments, compounds include those of Formula I, Ia, Ib, Ic, or Id, or pharmaceutically acceptable salts thereof, where G is:

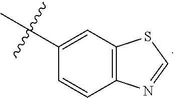 [SMILES: *c1ccc2ncsc2c1]

In certain embodiments, compounds include those of Formula I, Ia, Ib, Ic, or Id, or pharmaceutically acceptable salts thereof, where G is a 5-membered heteroaryl ring optionally substituted with one or two substituents, each independently selected from the group consisting of halo, halo-C₁-C₃ alkoxy, and halo-C₁-C₃ alkyl. In certain embodiments, compounds include those of Formula I, Ia, Ib, Ic, or Id, or pharmaceutically acceptable salts thereof, where G is a pyrazolyl substituted once with halo-C₁-C₃ alkyl. In certain embodiments, compounds include those of Formula I, Ia, Ib, Ic, or Id, or pharmaceutically acceptable salts thereof, where G is:

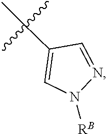

where R^{B} is selected from the group consisting of —CH₂CH₂F, —CH₂CHF₂, and —CH₂CF₃. In certain embodiments, compounds include those of Formula I, Ia, Ib, Ic, or Id, or pharmaceutically acceptable salts thereof, where R^{B} is —CH₂CHF₂.

In certain embodiments, compounds include those of Formula I, Ia, Ib, Ic, or Id, or pharmaceutically acceptable salts thereof, where R^{A} is halo. In certain embodiments, compounds include those of Formula I, Ia, Ib, Ic, or Id, or pharmaceutically acceptable salts thereof, where R^{A} is fluoro.

In certain embodiments, compounds include those of Formula I, Ia, Ib, Ic, or Id where m is 1. In certain embodiments, compounds include those of Formula I, Ia, Ib, Ic, or Id where m is 0.

In certain embodiments, compounds include those of Formula II, or pharmaceutically acceptable salts thereof:

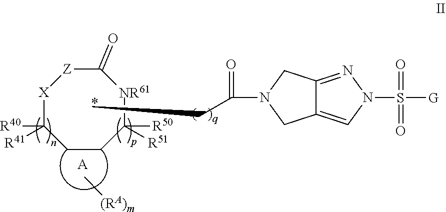

where all variables are as defined above.

In certain embodiments, compounds include those of Formula III, or pharmaceutically acceptable salts thereof:

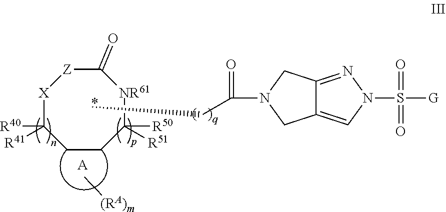

where all variables are as defined above.

In certain embodiments, compounds include those of Formula Ib2 or Ib3, or pharmaceutically acceptable salts thereof:

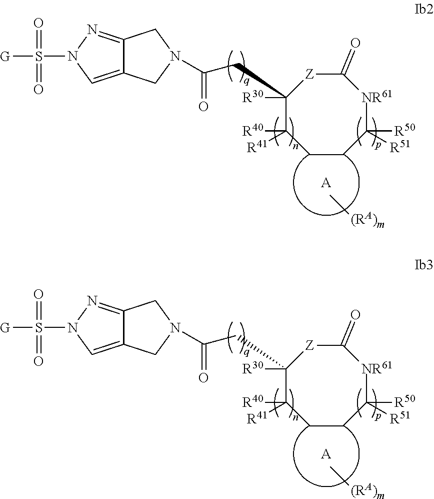

where all variables are as defined above.

In certain embodiments, compounds include those of Formula Id2 or Id3, or pharmaceutically acceptable salts thereof:

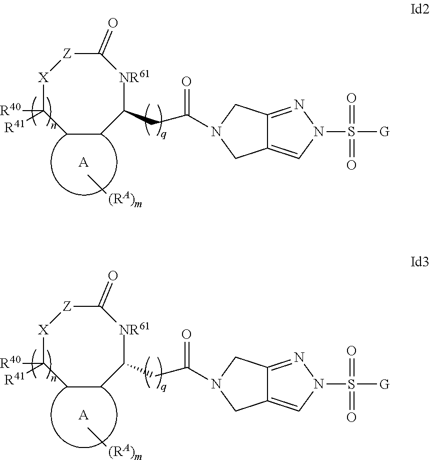

where all variables are as defined above.

In certain embodiments, the subject matter described herein includes the following compounds, or pharmaceutically acceptable salts thereof:

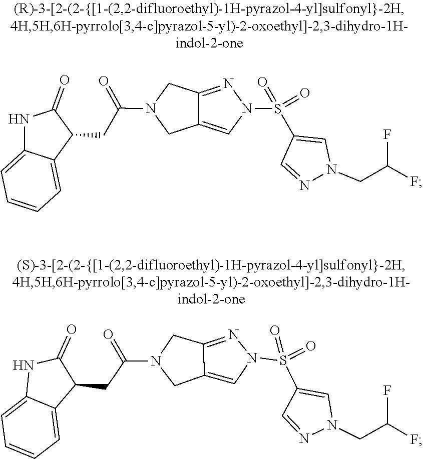

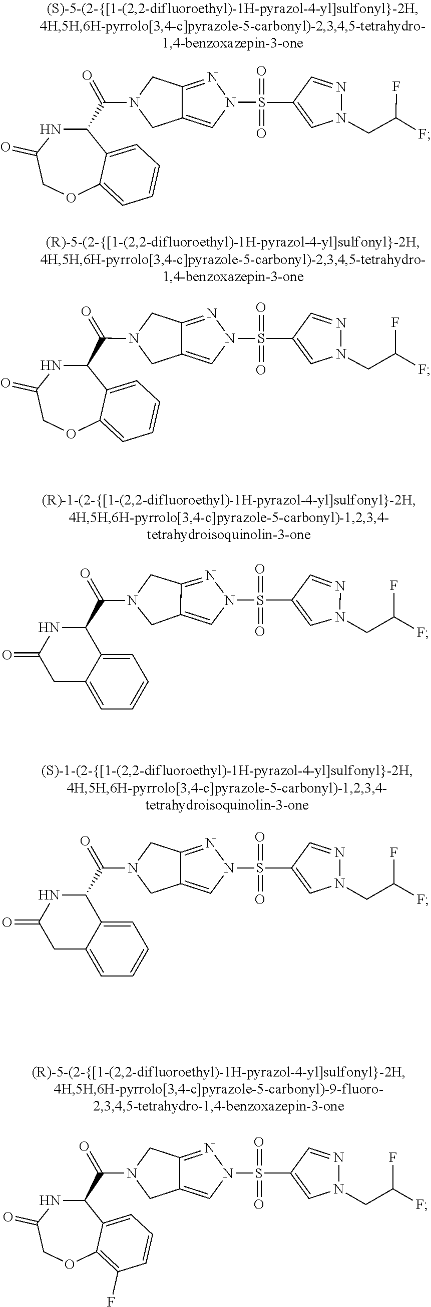 [SMILES: O=C1COc2ccccc2[C@@H](C(=O)N2Cc3cn(S(=O)(=O)c4cnn(CC(F)F)c4)nc3C2)N1 ; O=C1COc2ccccc2[C@H](C(=O)N2Cc3cn(S(=O)(=O)c4cnn(CC(F)F)c4)nc3C2)N1 ; O=C1Cc2ccccc2[C@H](C(=O)N2Cc3cn(S(=O)(=O)c4cnn(CC(F)F)c4)nc3C2)N1 ; O=C1Cc2ccccc2[C@@H](C(=O)N2Cc3cn(S(=O)(=O)c4cnn(CC(F)F)c4)nc3C2)N1 ; O=C1COc2c(F)cccc2[C@H](C(=O)N2Cc3cn(S(=O)(=O)c4cnn(CC(F)F)c4)nc3C2)N1]

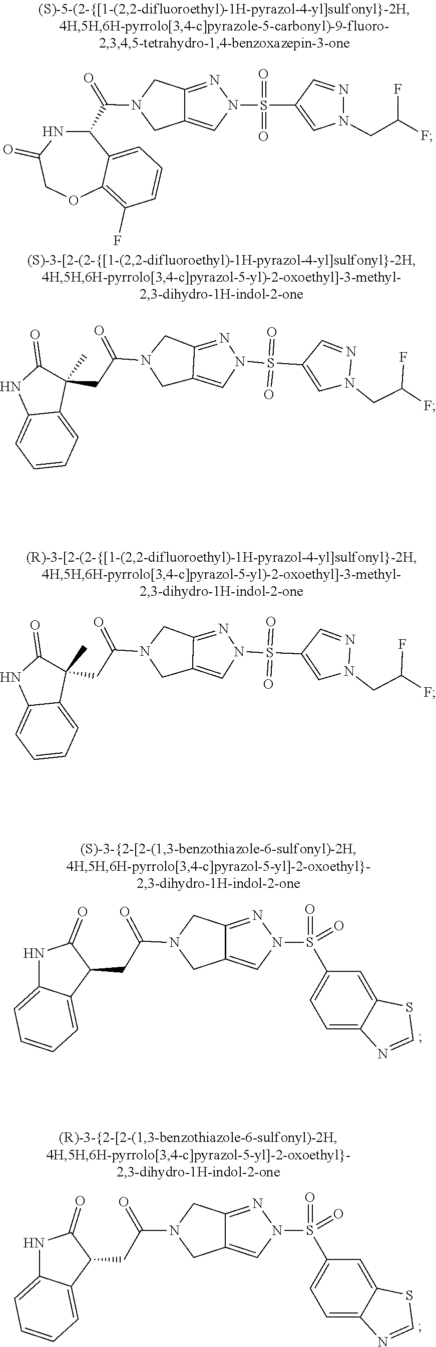

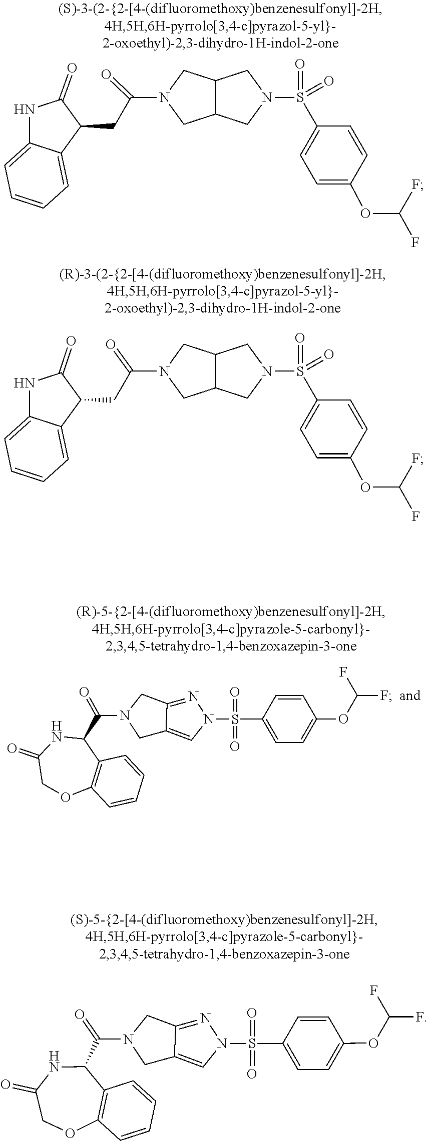

The subject matter described herein includes the following compounds in Table 1, stereoisomers, or pharmaceutically acceptable salts thereof.

TABLE 1

| | | | Mass |
|---|---|---|---|
| Cmpd | | | Found |
| No. | Structure | IUPAC Name | (M + 1) |
| 1 | 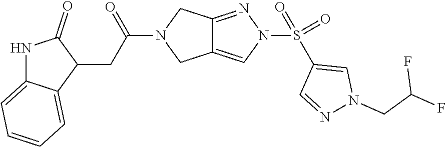 [SMILES: O=C1Nc2ccccc2C1CC(=O)N1Cc2cn(S(=O)(=O)c3cnn(CC(F)F)c3)nc2C1] | 3-[2-(2-{[1-(2,2-difluoroethyl)-1H- pyrazol-4-yl]sulfonyl}- 2H,4H,5H,6H-pyrrolo[3,4- c]pyrazol-5-yl)-2-oxoethyl]-2,3- dihydro-1H-indol-2-one | 477.12 |
| 2 | 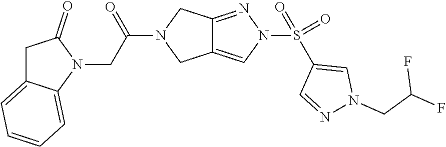 [SMILES: O=C(CN1C(=O)Cc2ccccc21)N1Cc2cn(S(=O)(=O)c3cnn(CC(F)F)c3)nc2C1] | 1-[2-(2-{[1-(2,2-difluoroethyl)-1H- pyrazol-4-yl]sulfonyl}- 2H,4H,5H,6H-pyrrolo[3,4- c]pyrazol-5-yl)-2-oxoethyl]-2,3- dihydro-1H-indol-2-one | 477.12 |
| 3 | 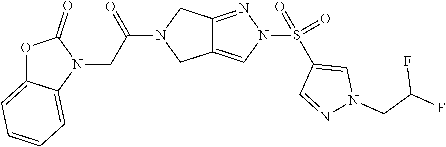 [SMILES: O=C(Cn1c(=O)oc2ccccc21)N1Cc2cn(S(=O)(=O)c3cnn(CC(F)F)c3)nc2C1] | 3-[2-(2-{[1-(2,2-difluoroethyl)-1H- pyrazol-4-yl]sulfonyl}- 2H,4H,5H,6H-pyrrolo[3,4- c]pyrazol-5-yl)-2-oxoethyl]-2,3- dihydro-1,3-benzoxazol-2-one | 479.03 |
| 4 | 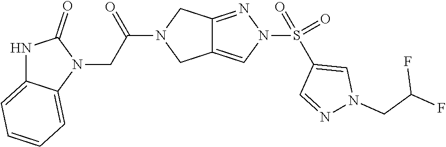 [SMILES: O=C(Cn1c(=O)[nH]c2ccccc21)N1Cc2cn(S(=O)(=O)c3cnn(CC(F)F)c3)nc2C1] | 1-[2-(2-{[1-(2,2-difluoroethyl)-1H- pyrazol-4-yl]sulfonyl}- 2H,4H,5H,6H-pyrrolo[3,4- c]pyrazol-5-yl)-2-oxoethyl]-2,3- dihydro-1H-1,3-benzodiazol-2-one | 478.12 |
| 5 | 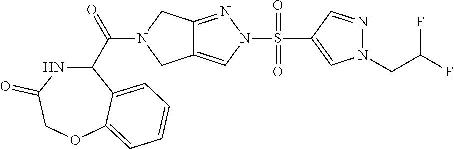 [SMILES: O=C1COc2ccccc2C(C(=O)N2Cc3cn(S(=O)(=O)c4cnn(CC(F)F)c4)nc3C2)N1] | 5-(2-{[1-(2,2-difluoroethyl)-1H- pyrazol-4-yl]sulfonyl}- 2H,4H,5H,6H-pyrrolo[3,4- c]pyrazole-5-carbonyl)-2,3,4,5- tetrahydro-1,4-benzoxazepin-3-one | 493.1 |
| 6 | 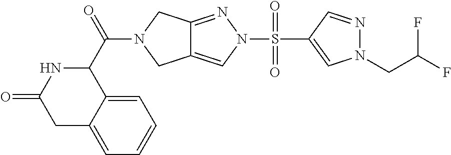 [SMILES: O=C1Cc2ccccc2C(C(=O)N2Cc3cn(S(=O)(=O)c4cnn(CC(F)F)c4)nc3C2)N1] | 1-(2-{[1-(2,2-difluoroethyl)-1H- pyrazol-4-yl]sulfonyl}- 2H,4H,5H,6H-pyrrolo[3,4- c]pyrazole-5-carbonyl)-1,2,3,4- tetrahydroisoquinolin-3-one | 477.0 |
| 7 | 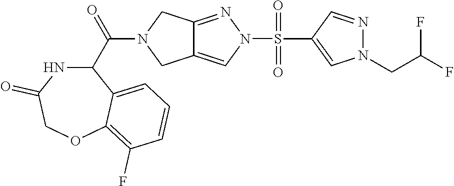 [SMILES: O=C1COc2c(F)cccc2C(C(=O)N2Cc3cn(S(=O)(=O)c4cnn(CC(F)F)c4)nc3C2)N1] | 5-(2-{[1-(2,2-difluoroethyl)-1H- pyrazol-4-yl]sulfonyl}- 2H,4H,5H,6H-pyrrolo[3,4- c]pyrazole-5-carbonyl)-9-fluoro- 2,3,4,5-tetrahydro-1,4- benzoxazepin-3-one | 511.0 |
| 8 | 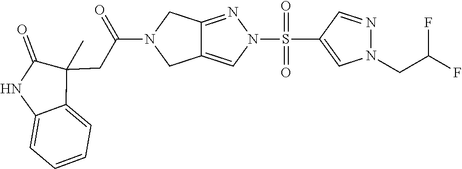 [SMILES: CC1(CC(=O)N2Cc3cn(S(=O)(=O)c4cnn(CC(F)F)c4)nc3C2)C(=O)Nc2ccccc21] | 3-[2-(2-{[1-(2,2-difluoroethyl)-1H- pyrazol-4-yl]sulfonyl}- 2H,4H,5H,6H-pyrrolo[3,4- c]pyrazol-5-yl)-2-oxoethyl]-3- methyl-2,3-dihydro-1H-indol-2-one | 491.0 |
| 9 | 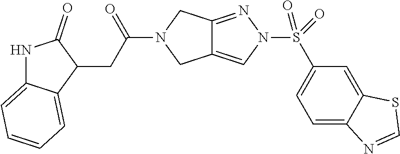 [SMILES: O=C1Nc2ccccc2C1CC(=O)N1Cc2cn(S(=O)(=O)c3ccc4ncsc4c3)nc2C1] | 3-{2-[2-(1,3-benzothiazole-6- sulfonyl)-2H,4H,5H,6H- pyrrolo[3,4-c]pyrazol-5-yl]-2- oxoethyl}-2,3-dihydro-1H-indol-2- one | 480.65 |
| 10 | 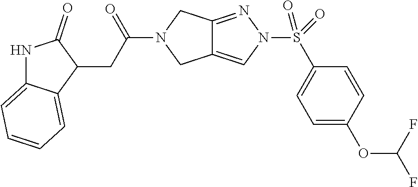 [SMILES: O=C1Nc2ccccc2C1CC(=O)N1Cc2cn(S(=O)(=O)c3ccc(OC(F)F)cc3)nc2C1] | 3-(2-{2-[4- (difluoromethoxy)benzenesulfonyl]- 2H,4H,5H,6H-pyrrolo[3,4- c]pyrazol-5-yl}-2-oxoethyl)-2,3- dihydro-1H-indol-2-one | 489.8 |
| 11 | 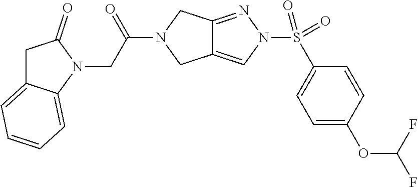 [SMILES: O=C(CN1C(=O)Cc2ccccc21)N1Cc2cn(S(=O)(=O)c3ccc(OC(F)F)cc3)nc2C1] | 1-(2-{2-[4- (difluoromethoxy)benzenesulfonyl]- 2H,4H,5H,6H-pyrrolo[3,4- c]pyrazol-5-yl}-2-oxoethyl)-2,3- dihydro-1H-indol-2-one | 489.3 |
| 12 | 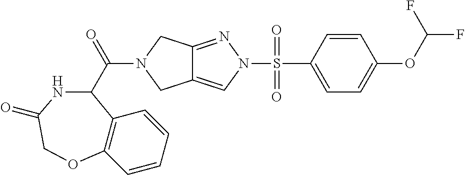 [SMILES: O=C1COc2ccccc2C(C(=O)N2Cc3cn(S(=O)(=O)c4ccc(OC(F)F)cc4)nc3C2)N1] | 5-{2-[4- (difluoromethoxy)benzenesulfonyl]- 2H,4H,5H,6H-pyrrolo[3,4- c]pyrazole-5-carbonyl}-2,3,4,5- tetrahydro-1,4-benzoxazepin-3-one | 505.1 |

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In certain embodiments, the subject matter disclosed herein is directed to a method of activating PKR and/or PKM2, including methods of treating a disease or disorder in a subject by administering a therapeutically effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I, Ia, Ib, Ib2, Ib3, Ic, Id, Id2, Id3, II, or III. In certain embodiments, the disease or disorder is selected from the group consisting of PKD (pyruvate kinase deficiency), SCD (e.g., sickle cell anemia), and thalassemia (e.g., beta-thalassemia).

In certain embodiments, the subject matter disclosed herein is directed to a method of treating a subject afflicted with a disease associated with decreased activity of PKR and/or PKM2, comprising administering to the subject an effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I, Ia, Ib, Ib2, Ib3, Ic, Id, Id2, Id3, II, or III. In certain embodiments, the disease associated with decreased activity of PKR is selected from the group consisting of hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Komzweig syndrome), paroxysmal nocturnal hemoglobinuria, acquired hemolytic anemia (e.g., congenital anemias (e.g., enzymopathies)), and anemia of chronic diseases.

In certain embodiments, the subject matter described herein is directed to a method of treating a disease or disorder associated with modulation of PKR and/or PKM2 in a subject, comprising administering to the subject an effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I, Ia, Ib, Ib2, Ib3, Ic, Id, Id2, Id3, II, or III.

In certain embodiments, the subject matter described herein is directed to a method of treating cancer in a subject in need thereof, comprising administering an effective amount of a compound of Formula I, Ia, Ib, Ib2, Ib3, Ic, Id, Id2, Id3, II, or III, a pharmaceutically acceptable salt thereof, or a pharmaceutical composition comprising a compound of Formula I, Ia, Ib, Ib2, Ib3, Ic, Id, Id2, Id3, II, or III. In certain embodiments, the cancer is selected from the group consisting of bladder cancer, breast cancer (e.g., ductal carcinoma), cervical cancer (e.g., squamous cell carcinoma), colorectal cancer (e.g., adenocarcinoma), esophageal cancer (e.g., squamous cell carcinoma), gastric cancer (e.g., adenocarcinoma, medulloblastoma, colon cancer, choriocarcinoma, squamous cell carcinoma), head and neck cancer, hematologic cancer (e.g., acute lymphocytic anemia, acute myeloid leukemia, acute lymphoblastic B cell leukemia, anaplastic large cell lymphoma, B-cell lymphoma, Burkitt's lymphoma, chronic lymphocytic leukemia, chronic eosinophillic leukemia/hypereosinophillic syndrome, chronic myeloid leukemia, Hodgkin's lymphoma, mantle cell lymphoma, multiple myeloma, T-cell acute lymphoblastic leukemia), lung cancer (e.g., bronchioloalveolar adenocarcinoma, mesothelioma, mucoepidermoid carcinoma, small-cell lung cancer, non-small cell lung cancer, adenocarcinoma, squamous cell carcinoma), liver cancer (e.g., hepatocellular carcinoma), lymphoma, neurological cancer (e.g., glioblastoma, neuroblastoma, neuroglioma), ovarian (e.g., adenocarcinoma), pancreatic cancer (e.g., ductal carcinoma), prostate cancer (e.g., adenocarcinoma), renal cancer (e.g., renal cell carcinoma, clear cell renal carcinoma), sarcoma (e.g., chondrosarcoma, Ewings sarcoma, fibrosarcoma, multipotential sarcoma, osteosarcoma, rhabdomyosarcoma, synovial sarcoma), skin cancer (e.g., melanoma, epidermoid carcinoma, squamous cell carcinoma), thyroid cancer (e.g., medullary carcinoma), and uterine cancer. In a preferred embodiment, the cancer is lung cancer.

In certain embodiments, compounds described herein, or pharmaceutically acceptable salts thereof, are useful as inhibitors of ubiquitin specific peptidase 9X (USP9X). USP9X inhibiting compounds are useful in the treatment of diseases and disorders associated with modulation of USP9X, such as cancer. See, for example WO/2020/061261 or WO/2021/055668.

In certain embodiments, the methods of administering and treating described herein further comprise co-administration of one or more additional pharmaceutically active compounds.

In a combination therapy, the pharmaceutically active compounds can be administered at the same time, in the same formulation, or at different times. Such combination therapy comprises co-administration of a compound of Formula I, Ia, Ib, Ib2, Ib3, Ic, Id, Id2, Id3, II, or III, or a pharmaceutically acceptable salt thereof with at least one additional pharmaceutically active compound. Combination therapy in a fixed dose combination therapy comprises co-administration of a compound of Formula I, Ia, Ib, Ib2, Ib3, Ic, Id, Id2, Id3, II, or III, or a pharmaceutically acceptable salt thereof with at least one additional pharmaceutically active compound in a fixed-dose formulation. Combination therapy in a free dose combination therapy comprises co-administration of a compound of Formula I, Ia, Ib, Ib2, Ib3, Ic, Id, Id2, Id3, II, or III, or a pharmaceutically acceptable salt thereof and at least one additional pharmaceutically active compound in free doses of the respective compounds, either by simultaneous administration of the individual compounds or by sequential use of the individual compounds over a period of time.

### V. Methods of Preparing Compounds of Formula I, Ia, Ib, Ib2, Ib3, Ic, Id, Id2, Id3, II, or III and Pharmaceutically Acceptable Salts Thereof

The starting materials and reagents used in preparing the compounds described herein are either available from commercial suppliers such as Sigma-Aldrich Chemical Co., (Milwaukee, Wis.), Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I, Ia, Ib, Ib2, Ib3, Ic, Id, Id2, Id3, II, or III may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### General Synthetic Schemes

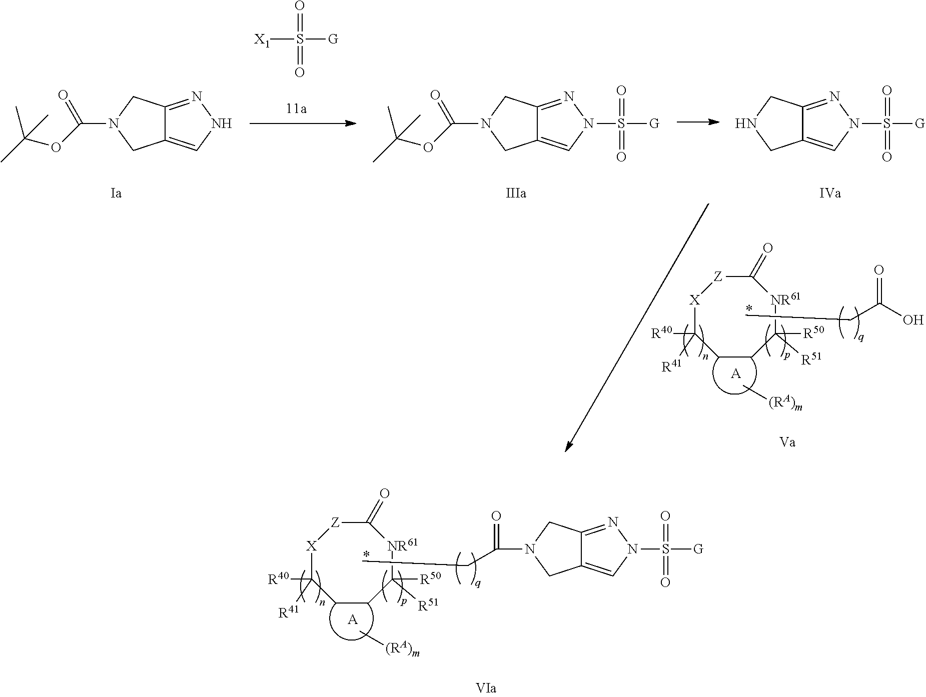

Compound Ia, which is commercially available or can be prepared by methods well known in the art, is treated with sodium hydride followed by a sulfonyl halide of formula IIa, to provide a compound of formula IIIa where G is as defined herein. For sulfonyl halides of formula IIa, X₁ₐ is halo, such as chloro, bromo, or iodo; and G is as defined herein. Removal of the protecting group of compound formula IIIa leads to the amine compound of formula IVa.

Reaction of compound of formula IVa with a carboxylic acid of formula Va, using amide coupling conditions well known in the art, gives a compound of formula VIa. Compounds of formula Va are either commercially available or can be prepared by methods well known in the art.

The subject matter described herein includes but is not limited to the following embodiments:

### A.1 A Compound of Formula I:

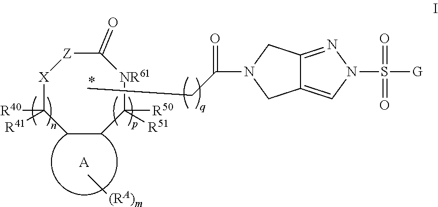

or a pharmaceutically acceptable salt thereof;
wherein,
   m is 0, 1, or 2;
   Ring A is a fused phenyl or 5- or 6-membered heteroaryl;
   R^{A}, in each instance, is independently selected from the group consisting of C₁-C₃ alkyl, C₁-C₃ alkoxy, hydroxy, halo-C₁-C₃ alkyl, halo-C₁-C₃ alkoxy, and halo;
   Z is a bond or CR²⁰R²¹;
      wherein, R²⁰ and R²¹ are each independently hydrogen or C₁-C₃ alkyl;
   X is NR³⁰, O or CR³⁰R³¹;
      wherein, R³⁰ and R³¹ are each independently hydrogen or C₁-C₃ alkyl;
   R⁴⁰, R⁴¹, R⁵⁰, R⁵¹ and R⁶¹ are each independently hydrogen or C₁-C₃ alkyl;
   n is 0 or 1;
   p is 0 or 1;
   * is an attachment point at any one of R²⁰, R²¹, R³⁰, R³¹, R⁴⁰, R⁴¹, R⁵⁰, R⁵¹ and R⁶¹;
   q is 0 or 1; and
   G is an optionally substituted heteroaryl, aryl, cycloalkyl, or heterocyclyl ring.

### A.2 A Compound of Formula I:

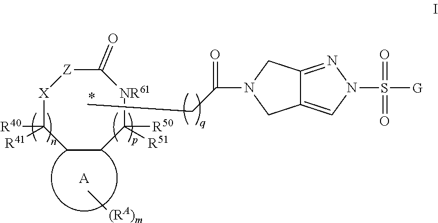

or a pharmaceutically acceptable salt thereof;
wherein,
   m is 0, 1, or 2;
   Ring A is a fused phenyl or 5- or 6-membered heteroaryl;
   R^{A}, in each instance, is independently selected from the group consisting of C₁-C₃ alkyl, C₁-C₃ alkoxy, hydroxy, halo-C₁-C₃ alkyl, halo-C₁-C₃ alkoxy, and halo;
   Z is a bond or CR²⁰R²¹;
      wherein, R²⁰ and R²¹ are each independently hydrogen or C₁-C₃ alkyl;
   X is NR³⁰, O or CR³⁰R³¹;
   wherein, R³⁰ and R³¹ are each independently hydrogen or C₁-C₃ alkyl;
   R⁴⁰, R⁴¹, R⁵⁰, R⁵¹ and R⁶¹ are each independently hydrogen or C₁-C₃ alkyl;
   n is 0 or 1;
   p is 0 or 1;
   * is an attachment point at any one of R²⁰, R²¹, R³⁰, R³¹, R⁴⁰, R⁴¹, R⁵⁰, R⁵¹ and R⁶¹;
   q is 0 or 1;
   G is:
      a) a 5-membered heteroaryl ring optionally substituted with one or two substituents, each independently selected from the group consisting of halo, C₁-C₃ alkyl, C₁-C₃ alkoxy, halo-C₁-C₃ alkoxy, and halo-C₁-C₃ alkyl;
   or,
      b)

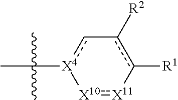

wherein,
   [symbol] represents a single or double bond;
   X⁴ is selected from the group consisting of N, C and CH;
   X¹⁰ and X¹¹ are each independently selected from the group consisting of N, CH₂, and CH, provided that only one of X¹⁰ and X¹¹ can be N;
   R¹ and R² are each independently selected from the group consisting of hydrogen, C₁-C₃ alkyl, amino, halo, halo-C₁-C₃ alkyl, halo-C₁-C₃ alkoxy, C₁-C₃ alkoxy, hydroxy, —O—R^{aa}, —(C₁-C₃ alkoxy)-R^{aa}, and C₃-C₇ cycloalkyl;
   R^{aa} is 4- to 7-membered heterocyclyl or C₃-C₇ cycloalkyl;
   or,
   R¹ and R², together with the carbon to which each is attached, form a 5- or 6-membered heteroaryl or 5- to 7-membered heterocyclyl containing one or two heteroatoms;
   wherein said heteroaryl or heterocyclyl comprising R¹ and R² is optionally substituted with one or two substituents, each independently selected from the group consisting of C₁-C₃ alkyl, halo-C₁-C₃ alkyl, C₁-C₃ alkoxy, halo, and halo-C₁-C₃ alkoxy.
1. A compound of Formula I:

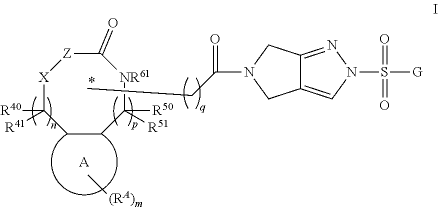

or a pharmaceutically acceptable salt thereof;
wherein,
   m is 0, 1, or 2;
   Ring A is a fused phenyl or 5- or 6-membered heteroaryl;
   R^{A}, in each instance, is independently selected from the group consisting of C₁-C₃ alkyl, C₁-C₃ alkoxy, hydroxy, halo-C₁-C₃ alkyl, halo-C₁-C₃ alkoxy, and halo;
   Z is a bond or CR²⁰R²¹;
      wherein, R²⁰ and R²¹ are each independently hydrogen or C₁-C₃ alkyl;
   X is NR³⁰, O or CR³⁰R³¹;
      wherein, R³⁰ and R³¹ are each independently hydrogen or C₁-C₃ alkyl;
   R⁴⁰, R⁴¹, R⁵⁰, R⁵¹ and R⁶¹ are each independently hydrogen or C₁-C₃ alkyl;
   n is 0 or 1;
   p is 0 or 1;
   * is an attachment point at any one of R²⁰, R²¹, R³⁰, R³¹, R⁴⁰, R⁴¹, R⁵⁰, R⁵¹ and R⁶¹;
   q is 0 or 1;
   G is:
      a) a 5-membered heteroaryl ring optionally substituted with one or two substituents, each independently selected from the group consisting of halo, C₁-C₃ alkyl, C₁-C₃ alkoxy, halo-C₁-C₃ alkoxy, and halo-C₁-C₃ alkyl;
   or,
      b)

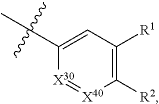

wherein,
X³⁰ and X⁴⁰ are each independently N or CH, provided that only one of X³⁰ and X⁴⁰ can be N;
R¹ and R² are each independently selected from the group consisting of hydrogen, C₁-C₃ alkyl, amino, halo, halo-C₁-C₃ alkyl, halo-C₁-C₃ alkoxy, C₁-C₃ alkoxy, hydroxy, —O—R^{aa}, —(C₁-C₃ alkoxy)-R^{aa}, and C₃-C₇ cycloalkyl;
R^{aa} is 4- to 7-membered heterocyclyl or C₃-C₇ cycloalkyl;
or,
R¹ and R², together with the carbon to which each is attached, form a 5- or 6-membered heteroaryl or 5- to 7-membered heterocyclyl containing one or two heteroatoms;
wherein said heteroaryl or heterocyclyl comprising R¹ and R² is optionally substituted with one or two substituents, each independently selected from the group consisting of C₁-C₃ alkyl, halo-C₁-C₃ alkyl, C₁-C₃ alkoxy, halo, and halo-C₁-C₃ alkoxy.
2. The compound of embodiment 1, wherein ring A is phenyl and the compound is of Formula Ia:

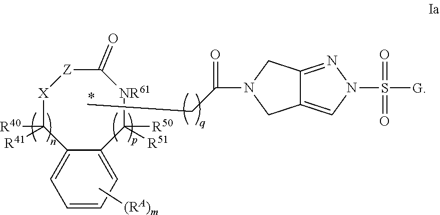

3. The compound of embodiment 1 or 2, wherein Z is a bond.
4. The compound of embodiment 1 or 2, wherein Z is CR²⁰R²¹.
5. The compound of embodiment 4, wherein R²⁰ and R²¹ are each hydrogen.
6. The compound of any one of embodiments 1-5, wherein X is CR³⁰R³¹.
7. The compound of embodiment 6, wherein R³⁰ is hydrogen.
8. The compound of embodiment 6, wherein R³⁰ is methyl.
9. The compound of embodiment 6, wherein R³¹ is * and the compound is of Formula Ib:

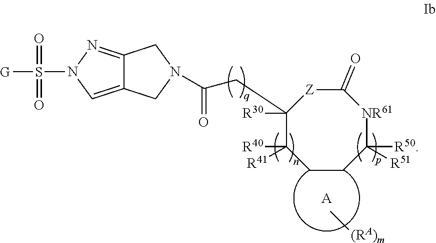

10. The compound of embodiment 6, wherein R³¹ is hydrogen.
11. The compound of any one of embodiments 1-5, wherein X is O.
12. The compound of any one of embodiments 1-5, wherein X is NR³⁰.
13. The compound of embodiment 12, wherein R³⁰ is hydrogen.
14. The compound of any one of embodiments 1-13, wherein R⁶¹ is hydrogen.
15. The compound of any one of embodiments 1-8 or 10-13, wherein R⁶¹ is * and the compound is of Formula Ic:

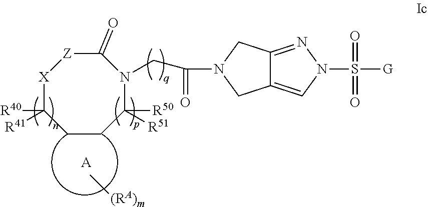

16. The compound of any one of embodiments 1-15, wherein n is 0.
17. The compound of any one of embodiments 1-8, 10-14, or 16, wherein p is 1, R⁵⁰ is hydrogen and R⁵¹ is * and the compound is of Formula Id:

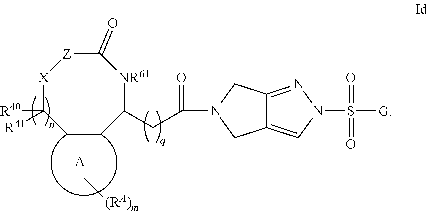

18. The compound of any one of embodiments 1-16, wherein p is 0.
19. The compound of embodiment 1 or 2, wherein R³¹ is * and the compound is of Formula Ib:

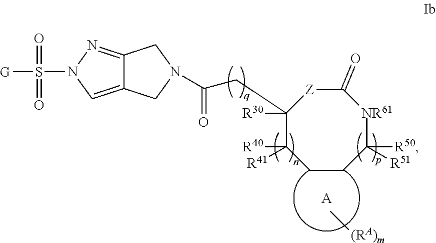

wherein,
   Z is a bond;
   R³⁰ is hydrogen or methyl;
   R⁶¹ is hydrogen;
   n is 0; and
   p is 0.
20. The compound of embodiment 1 or 2, wherein R⁶¹ is * and the compound is of Formula IC:

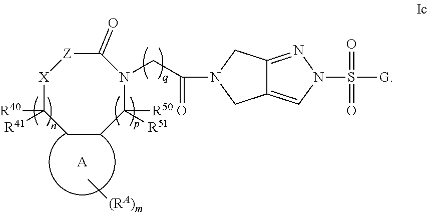

wherein,
   Z is a bond;
   X is O, NH, or CH₂;
   n is 0;
   and p is 0.
21. The compound of embodiment 1 or 2, wherein p is 1, R⁵⁰ is hydrogen, R⁵¹ is * and the compound is of Formula Id:

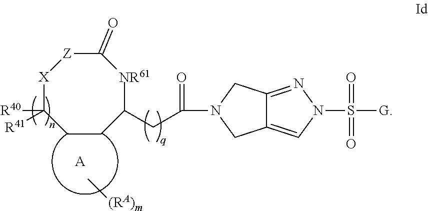

wherein,
   Z is CH₂;
   X is O;
   n is 0; and
   R⁶¹ is hydrogen.
22. The compound of embodiment 1 or 2, wherein p is 1, R⁵⁰ is hydrogen, R⁵¹ is * and the compound is of Formula Id:

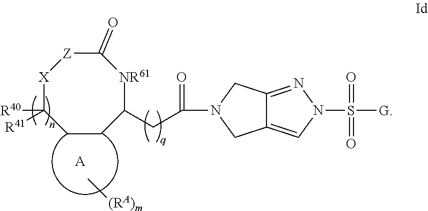

wherein,
   Z is a bond;
   X is CH₂;
   n is 0; and,
   R⁶¹ is hydrogen.
23. The compound of any one of embodiments 1-22, wherein q is 0.
24. The compound of any one of embodiments 1-22, wherein q is 1.
25. The compound of any one of embodiments 1-24, wherein G is phenyl, wherein R¹ is hydrogen and R² is halo-C₁-C₃ alkoxy.
26. The compound of embodiment 25, wherein R² is —OCH₂F, —OCHF₂, or —OCF₃.
27. The compound of embodiment 26, wherein R² is —OCHF₂.
28. The compound of any one of embodiments 1-24, wherein G is phenyl, wherein R¹ and R², together with the carbon to which each is attached, form a 5-membered heteroaryl.
29. The compound of embodiment 28, wherein R¹ and R², together with the carbon to which each is attached, form a thiazolyl.
30. The compound of embodiment 28 or 29, wherein G is:

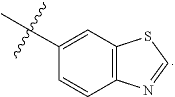 [SMILES: *c1ccc2ncsc2c1]

31. The compound of any one of embodiments 1-24, wherein G is a 5-membered heteroaryl ring optionally substituted with one or two substituents, each independently selected from the group consisting of halo, halo-C₁-C₃ alkoxy, and halo-C₁-C₃ alkyl.
32. The compound of embodiment 31, wherein G is a pyrazolyl substituted once with halo-C₁-C₃ alkyl.
33. The compound of embodiment 32, wherein G is:

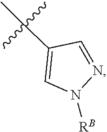

wherein,
   R^{B} is selected from the group consisting of —CH₂CH₂F, —CH₂CHF₂, and —CH₂CF₃.
34. The compound of embodiment 33, wherein R^{B} is —CH₂CHF₂.
35. The compound of any one of embodiments 1-34, wherein R^{A} is halo.
36. The compound of embodiment 35, wherein R^{A} is fluoro.
37. The compound of any one of embodiments 1-36, wherein m is 0.
38. The compound of any one of embodiments 1-36, wherein m is 1.
39. The compound of embodiment 1, wherein the compound is selected from Table 1, or a pharmaceutically acceptable salt thereof.
40. A pharmaceutical composition comprising a compound of any one of embodiments 1-39 or a pharmaceutically acceptable salt thereof, and a pharmaceutically acceptable excipient.
41. A method of treating a disease or disorder associated with modulation of pyruvate kinase (PKR) and/or PKM2 in a subject, comprising administering to the subject an effective amount of a compound of any one of embodiments 1-39 or the pharmaceutical composition of embodiment 40.
42. A method of activating PKR and/or PKM2 in a subject, comprising administering to the subject an effective amount of a compound of any one of embodiments 1-39 or the pharmaceutical composition of embodiment 40.
43. A method of treating a subject afflicted with a disease associated with decreased activity of PKR and/or PKM2, comprising administering to the subject an effective amount of a compound of any one of embodiments 1-39 or the pharmaceutical composition of embodiment 40.
44. The method of embodiment 43, wherein the disease is selected from the group consisting of sickle cell disease, sickle cell anemia, thalassemia, hereditary non-spherocytic hemolytic anemia, hemolytic anemia, hereditary spherocytosis, hereditary elliptocytosis, abetalipoproteinemia, paroxysmal nocturnal hemoglobinuria, acquired hemolytic, and anemia of chronic diseases.

The following examples are offered by way of illustration and not by way of limitation.

### EXAMPLES

### I. Synthetic Examples

Several intermediates used in the synthetic preparations of the compounds described herein are provided below:

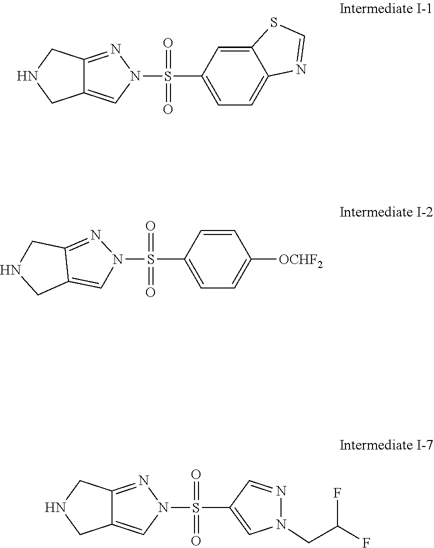

### Example 1.1

### Synthesis of 6-{2H,4H,5H,6H-pyrrolo[3,4-c]pyrazole-2-sulfonyl}-1,3-benzothiazole (Intermediate I-1)

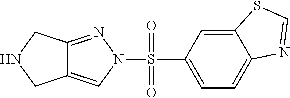 [SMILES: O=S(=O)(c1ccc2ncsc2c1)n1cc2c(n1)CNC2]

### Step 1

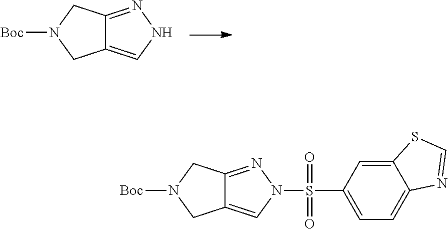 [SMILES: CC(C)(C)OC(=O)N1Cc2c[nH]nc2C1 ; CC(C)(C)OC(=O)N1Cc2cn(S(=O)(=O)c3ccc4ncsc4c3)nc2C1]

To a suspension of sodium hydride (0.70 g; 60%, 17.41 mmol; 1.30 eq.) in dry THF (15 mL) in an ice/water bath under an N₂ atmosphere was added a solution of N-{2H,4H,5H,6H-pyrrolo[3,4-c]pyrazol-5-yl}(tert-butoxy)formamide (3.0 g; 13.40 mmol; 1.00 eq.) in THF (15 mL) over 5 min. The mixture was allowed to stir in the ice bath for 30 min. A solution of 1,3-benzothiazole-6-sulfonyl chloride (3 130.39 mg; 13.40 mmol; 1.00 eq.) in THF (15 mL) was added dropwise over 5 min. The mixture was allowed to stir in the ice bath for 45 min, and at rt for 3 h subsequently. To the resulting mixture under N₂ atmosphere was added aq. NH₄Cl solution (15 mL) and HOAc to adjust the pH to ˜5. The organic layer was separated, and the aqueous layer was back extracted with 10% MeOH/CH₂Cl₂ (15) thrice. The combined organic solutions were washed with water and brine. Removal of the volatiles under reduced pressure provided the desired crude product of tert-butyl 2-(1,3-benzothiazole-6-sulfonyl)-2H,4H,5H,6H-pyrrolo[3,4-c]pyrazole-5-carboxylate as a pale yellow solid (5.1 g, 90%), which was directly used in the next step. ¹H NMR (DMSO-d₆) δ: 9.70 (s, 1H), 9.02 (s, 1H), 8.37-8.17 (m, 2H), 8.03 (dt, J=8.7, 1.6 Hz, 1H), 4.30 (dd, J=9.8, 3.8 Hz, 4H), 1.39 (s, 9H); LCMS (ES) [M+1]⁺ m/z 407.3.

### Step 2

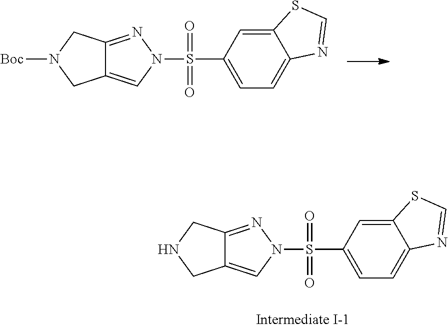 [SMILES: CC(C)(C)OC(=O)N1Cc2cn(S(=O)(=O)c3ccc4ncsc4c3)nc2C1 ; O=S(=O)(c1ccc2ncsc2c1)n1cc2c(n1)CNC2]

To a solution of tert-butyl 2-(1,3-benzothiazole-6-sulfonyl)-2H,4H,5H,6H-pyrrolo[3,4-c]pyrazole-5-carboxylate (4.1 g; 10.0 mol; 1.00 eq.) in CH₂Cl₂ (10 mL) at rt was added a solution of HCl in dioxane (15 mL; 4.00 mol/L; 60 mmol; 6.00 eq.). After stirring at rt for 75 min, the resulting precipitate was filtered, washed with cold CH₂Cl₂ (15) twice, and left on high vacuum overnight to provide the hydrogen chloride salt of the title compound as a white solid (2.86, 93%). LCMS (ES) [M+1]⁺ m/z 307.3; ¹H NMR (DMSO-d₆) δ: 10.22 (s, 2H), 9.72 (s, 1H), 9.07 (d, J=2.0 Hz, 1H), 8.45-8.20 (m, 2H), 8.07 (dd, J=8.7, 2.1 Hz, 1H), 4.27 (d, J=7.1 Hz, 4H).

### Example 1.2

### Synthesis of 2-[4-(difluoromethoxy)benzenesulfonyl]-2H,4H,5H,6H-pyrrolo[3,4-c]pyrazole (Intermediate I-2)

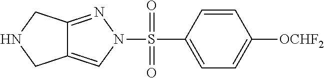 [SMILES: O=S(=O)(c1ccc(OC(F)F)cc1)n1cc2c(n1)CNC2]

The title compound was made from N-{2H,4H,5H,6H-pyrrolo[3,4-c]pyrazol-5-yl}(tert-butoxy)formamide following a procedure similar to that described for the synthesis of Intermediate I-1, except that 4-(difluoromethoxy)benzenesulfonyl chloride was used in the place of 1,3-benzothiazole-6-sulfonyl chloride (step 1). The crude product was purified by silica gel column with 0-10% MeOH/CH₂Cl₂(0.1% NH₄OH) as the eluent to provide the desired product as a white solid. LCMS (ES) [M+1]⁺ m/z 316.0.

### Example 1.3

### Synthesis of 1-(2,2-difluoroethyl)-4-{2H,4H,5H,6H-pyrrolo[3,4-c]pyrazole-2-sulfonyl}-1H-pyrazole (Intermediate I-7)

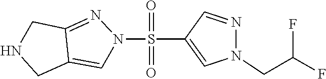

### Step 1

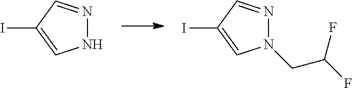 [SMILES: Ic1cn[nH]c1 ; FC(F)Cn1cc(I)cn1]

Into a 500-mL 3-necked round-bottom flask, was placed 4-iodopyrazole (20.00 g, 103.11 mmol, 1.00 eq.) in DMF (250.00 mL) followed by the addition of NaH (4.95 g, 206.21 mmol, 2.00 eq.), at 0° C. The resulting mixture was stirred for 0.5 h at room temperature, then 1,1-difluoro-2-iodoethane (29.69 g, 154.66 mmol, 1.50 eq.) was added dropwise with stirring at 0° C. The resulting solution was stirred overnight at room temperature, then it was quenched by the addition of water/ice. The resulting solution was extracted with 3×200 mL of ethyl acetate and the organic layers were combined and dried over anhydrous sodium sulfate and concentrated. The residue was applied onto a silica gel column and eluted with THF/PE (8%). This resulted in 21 g (78.94%) of 1-(2,2-difluoroethyl)-4-iodopyrazole as a colorless oil. LCMS (ES) [M+1]⁺ m/z: 259.

### Step 2

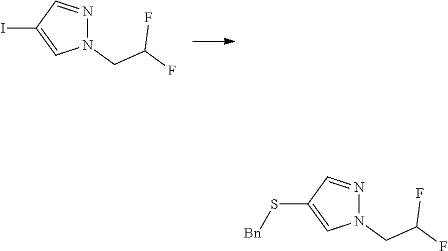 [SMILES: FC(F)Cn1cc(I)cn1 ; FC(F)Cn1cc(SCc2ccccc2)cn1]

Into a 500-mL round-bottom flask purged and maintained at room temperature in an atmosphere of nitrogen, was placed 1-(2,2-difluoroethyl)-4-iodopyrazole (21.00 g, 81.39 mmol, 1.00 eq.), benzyl mercaptan (30.33 g, 244.17 mmol, 3.00 eq.), dioxane (250.00 mL), DIEA (31.56 g, 244.19 mmol, 3.00 eq.), XantPhos (9.42 g, 16.28 mmol, 0.20 eq.) and Pd₂(dba)₃ (7.45 g, 8.14 mmol, 0.10 eq.). The resulting mixture was stirred overnight at 100° C., then it was cooled to room temperature and concentrated. The residue was applied onto a silica gel column and eluted with THF/PE (5%). This resulted in 14.4 g (69.57%) of 4-(benzylsulfanyl)-1-(2,2-difluoroethyl)pyrazole as a yellow oil. LCMS (ES) [M−1]⁺ m/z: 255.

Step 3

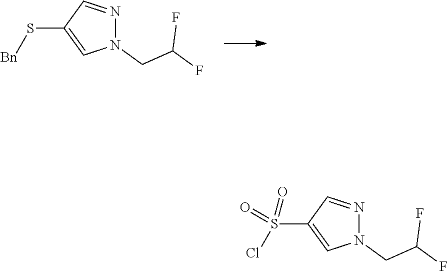 [SMILES: FC(F)Cn1cc(SCc2ccccc2)cn1 ; O=S(=O)(Cl)c1cnn(CC(F)F)c1]

Into a 250-mL round-bottom flask, was placed 4-(benzylsulfanyl)-1-(2,2-difluoroethyl)pyrazole (14.40 g, 56.63 mmol, 1.00 eq.), HOAc (180.00 mL), and H₂O (20.00 mL). This was followed by the addition of NCS (22.68 g, 169.85 mmol, 3.00 eq.), at 0° C. and the resulting solution was stirred for 1 h at room temperature. Then it was concentrated. The residue was applied onto a silica gel column and eluted with THF/PE (10%). This resulted in 11 g (84.24%) of 1-(2,2-difluoroethyl)pyrazole-4-sulfonyl chloride as a yellow oil. LCMS (ES) [M-Cl+OH-1]⁻ m/z: 211.

### Step 4

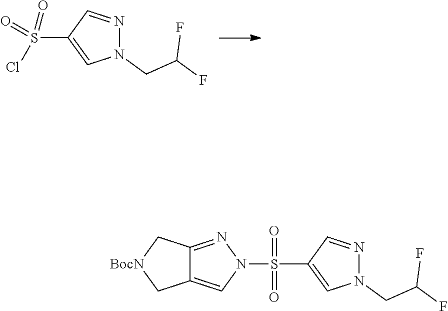 [SMILES: O=S(=O)(Cl)c1cnn(CC(F)F)c1 ; CC(C)(C)OC(=O)N1Cc2cn(S(=O)(=O)c3cnn(CC(F)F)c3)nc2C1]

Into a 250-mL 3-necked round-bottom flask, was placed tert-butyl 2H,4H,6H-pyrrolo[3,4-c]pyrazole-5-carboxylate (11.00 g, 52.57 mmol, 1.00 eq.) in THF (180.00 mL), followed by the addition of NaH (1.64 g, 68.34 mmol, 1.30 eq.), at 0° C. To this was then added 1-(2,2-difluoroethyl)pyrazole-4-sulfonyl chloride (13.34 g, 57.85 mmol, 1.10 eq.) dropwise with stirring at 0° C. and the resulting solution was stirred for 1 h at 0° C. The reaction was then quenched by the addition of 5 mL of HOAc and extracted with 3×200 mL of dichloromethane. The organic layers were combined, dried over anhydrous sodium sulfate, and concentrated. The residue was diluted with 200 mL of MTBE and the solids were collected by filtration. This resulted in 15.3 g (72.15%) of tert-butyl 2-[1-(2,2-difluoroethyl)pyrazol-4-ylsulfonyl]-4H,6H-pyrrolo[3,4-c]pyrazole-5-carboxylate as a white solid. LCMS (ES) [M+1]⁺ m/z: 404.

### Step 5

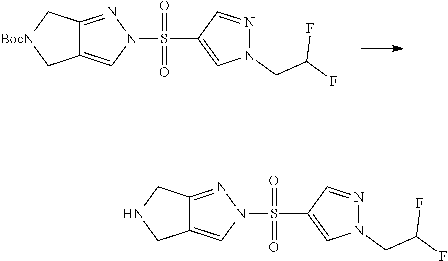 [SMILES: CC(C)(C)OC(=O)N1Cc2cn(S(=O)(=O)c3cnn(CC(F)F)c3)nc2C1 ; O=S(=O)(c1cnn(CC(F)F)c1)n1cc2c(n1)CNC2]

Into a 250-mL round-bottom flask, was placed tert-butyl 2-[1-(2,2-difluoroethyl)pyrazol-4-ylsulfonyl]-4H,6H-pyrrolo[3,4-c]pyrazole-5-carboxylate (15.30 g, 37.93 mmol, 1.00 eq.), DCM (200.00 mL), and lutidine (16.26 g, 151.71 mmol, 4.00 eq.) followed by the addition of TMSOTf (25.29 g, 113.78 mmol, 3.00 eq.) dropwise with stirring at 0° C. The resulting solution was stirred for 1 h at 0° C., then it was quenched by the addition of water/ice. The resulting mixture was concentrated, and the crude product (30 g) was purified by Prep-HPLC with the following conditions: Column, XBridge Prep C18 OBD Column, 19 cm, 150 mm, 5 um; mobile phase, Water (0.1% FA) and CAN (5% Phase B up to 20% in 11 min); Detector, 254. This resulted in the title compound (Intermediate-7) (10 g, 86.9%) as a white solid. LCMS (ES) [M+1]⁺ m/z: 304.

### Example 1.4

### Synthesis of 3-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1H-indol-2-one (Compound 1)

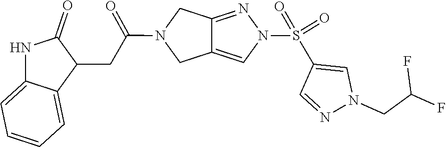 [SMILES: O=C1Nc2ccccc2C1CC(=O)N1Cc2cn(S(=O)(=O)c3cnn(CC(F)F)c3)nc2C1]

To a solution of 1-(2,2-difluoroethyl)-4-{2H,4H,5H,6H-pyrrolo[3,4-c]pyrazole-2-sulfonyl}-1H-pyrazole (Intermediate I-7, 86.00 mg; 0.28 mmol; 1.00 eq.) and (2-oxo-2,3-dihydro-1H-indol-3-yl)acetic acid (54.21 mg; 0.28 mmol; 1.00 eq.) in N,N-dimethylformamide (3.01 mL) were added Hunig's base (0.15 mL; 0.86 mol; 3.00 eq.) and HATU (107.82 mg; 0.28 mol; 1.00 eq.) and the reaction mixture was left stirring at RT overnight. The reaction mix was cooled to 0° C. and treated with dropwise addition of water. A precipitate formed that was removed by filtration. The crude solid was taken in DMSO and purified by prep HPLC using 0 to 80% acetonitrile in 0.1% aqueous formic acid. The fractions containing the product were combined and freeze dried to give 3-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1H-indol-2-one as a white solid (65 mg, 48.1%). ¹H NMR (400 MHz, DMSO-d₆) δ 10.34 (d, J=7.4 Hz, 1H), 8.80-8.66 (m, 1H), 8.21-8.05 (m, 1H), 7.20 (dt, J=13.9, 6.6 Hz, 1H), 7.14-7.04 (m, 1H), 6.90-6.80 (m, 1H), 6.77 (dd, J=7.2, 5.7 Hz, 1H), 6.58-6.20 (m, 1H), 5.03-4.89 (m, 1H), 4.78-4.46 (m, 4H), 4.42-4.26 (m, 1H), 3.70 (d, J=7.2 Hz, 1H), 3.08 (ddd, J=35.4, 17.0, 3.6 Hz, 1H), 2.79 (ddd, J=22.7, 17.0, 8.1 Hz, 1H). LCMS (ES) [M+1]⁺ m/z: 477.12.

### Example 1.5

### Synthesis of 1-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1H-indol-2-one (Compound 2)

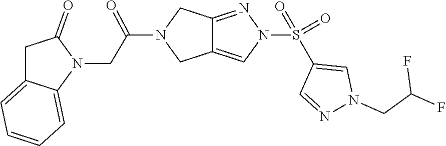 [SMILES: O=C(CN1C(=O)Cc2ccccc21)N1Cc2cn(S(=O)(=O)c3cnn(CC(F)F)c3)nc2C1]

The title compound was synthesized according to the procedure described in Example 1.4 for Compound 1 using 1-(2,2-difluoroethyl)-4-{2H,4H,5H,6H-pyrrolo[3,4-c]pyrazole-2-sulfonyl}-1H-pyrazole (Intermediate I-7, 120.00 mg; 0.40 mmol; 1.00 eq.) and 2-(2-oxo-2,3-dihydro-1H-indol-1-yl)acetic acid (75.64 mg; 0.40 mmol; 1.00 eq.) to give 1-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1H-indol-2-one as a white solid (51 mg, 27%). ¹H NMR (400 MHz, DMSO-d₆) δ 8.79 (dd, J=7.2, 0.7 Hz, 1H), 8.22-8.19 (m, 1H), 7.79 (d, J=20.1 Hz, 1H), 7.27-7.22 (m, 1H), 7.22-7.15 (m, 1H), 6.97 (td, J=7.5, 1.0 Hz, 1H), 6.91 (dd, J=7.8, 4.9 Hz, 1H), 6.59-6.21 (m, 1H), 5.18 (d, J=2.6 Hz, 1H), 4.80-4.54 (m, 6H), 4.36 (s, 1H), 3.60 (s, 2H). LCMS (ES) [M+1]⁺ m/z: 477.12.

### Example 1.6

### Synthesis of 3-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1,3-benzoxazol-2-one (Compound 3)

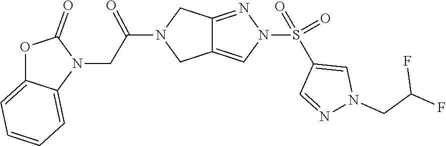 [SMILES: O=C(Cn1c(=O)oc2ccccc21)N1Cc2cn(S(=O)(=O)c3cnn(CC(F)F)c3)nc2C1]

The title compound was synthesized according to the procedure described in Example 1.4 for Compound 1 using 1-(2,2-difluoroethyl)-4-{2H,4H,5H,6H-pyrrolo[3,4-c]pyrazole-2-sulfonyl}-1H-pyrazole (Intermediate I-7, 120.00 mg; 0.40 mmol; 1.00 eq.) and (2-oxo-1,3-benzoxazol-3(2H)-yl)acetic acid (76.42 mg; 0.40 mmol; 1.00 eq.) to give 3-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1,3-benzoxazol-2-one as a white solid (28 mg, 14.8%). ¹H NMR (400 MHz, DMSO-d₆) δ 8.73 (dd, J=4.3, 0.7 Hz, 1H), 8.36-8.18 (m, 1H), 8.12 (dd, J=2.8, 0.7 Hz, 1H), 7.33 (dd, J=7.6, 1.2 Hz, 1H), 7.24-7.02 (m, 3H), 6.40 (ddt, J=57.9, 54.4, 3.5 Hz, 1H), 4.90-4.77 (m, 4H), 4.71 (tt, J=15.1, 3.2 Hz, 2H), 4.42 (d, J=15.9 Hz, 2H). LCMS (ES) [M+1]⁺ m/z: 479.03.

### Example 1.7

### Synthesis of 1-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1H-1,3-benzodiazol-2-one (Compound 4)

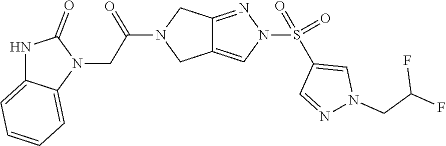 [SMILES: O=C(Cn1c(=O)[nH]c2ccccc21)N1Cc2cn(S(=O)(=O)c3cnn(CC(F)F)c3)nc2C1]

The title compound was synthesized according to the procedure described in Example 1.4 for Compound 1 using 1-(2,2-difluoroethyl)-4-{2H,4H,5H,6H-pyrrolo[3,4-c]pyrazole-2-sulfonyl}-1H-pyrazole (Intermediate I-7, 80.00 mg; 0.26 mmol; 1.00 eq.) and 2-(2-oxo-2,3-dihydro-1H-1,3-benzodiazol-1-yl)acetic acid (50.69 mg; 0.26 mmol; 1.00 eq.) to give 1-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-2,3-dihydro-1H-1,3-benzodiazol-2-one as a white solid (74 mg, 58.8%). ¹H NMR (400 MHz, DMSO-d₆) δ 10.84 (d, J=4.0 Hz, 1H), 8.80 (dd, J=5.7, 0.7 Hz, 1H), 8.21 (dd, J=2.4, 0.7 Hz, 1H), 7.80 (d, J=20.4 Hz, 1H), 7.06-6.86 (m, 4H), 6.58-6.19 (m, 1H), 5.18 (s, 1H), 4.85-4.64 (m, 6H), 4.36 (d, J=2.5 Hz, 1H). LCMS (ES) [M+1]⁺ m/z: 478.12.

### Example 1.8

### Synthesis of 5-(2-{[1-(2,2-difluoroethyl)-1H-pyrazol-4-yl]sulfonyl}-2H,4H,5H,6H-pyrrolo[3,4-c]pyrazole-5-carbonyl)-2,3,4,5-tetrahydro-1,4-benzoxazepin-3-one (Compound 5)

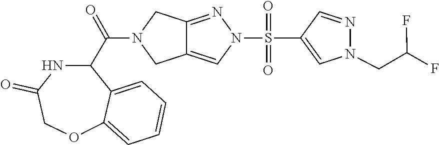 [SMILES: O=C1COc2ccccc2C(C(=O)N2Cc3cn(S(=O)(=O)c4cnn(CC(F)F)c4)nc3C2)N1]

### Step 1

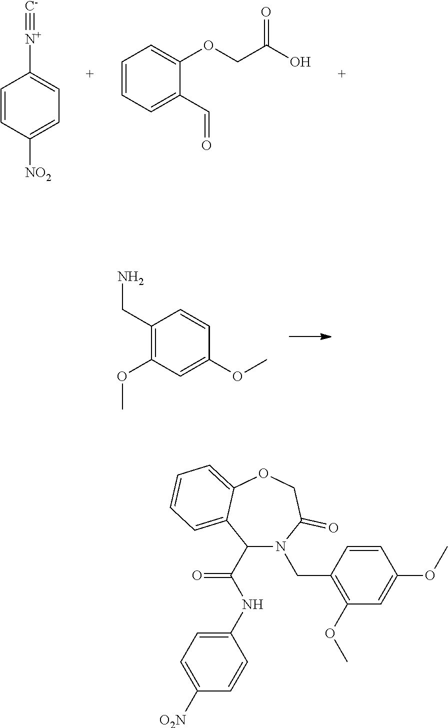 [SMILES: [C-]#[N+]c1ccc([N+](=O)[O-])cc1 ; O=Cc1ccccc1OCC(=O)O ; COc1ccc(CN)c(OC)c1 ; COc1ccc(CN2C(=O)COc3ccccc3C2C(=O)Nc2ccc([N+](=O)[O-])cc2)c(OC)c1]

2,4-Dimethoxybenzylamine (1.13 g, 6.8 mmol, 1.0 eq.) was added to a solution containing (2-formylphenoxy)acetic acid (1.22 g, 6.8 mmol, 1.0 eq.) in methanol (23 mL). After 10 minutes, 1-isocyano-4-nitrobenzene (1.00 g, 6.8 mmol, 1.0 eq.) in tetrahydrofuran (4.5 mL) was added all at once and the reaction was stirred for 24 hours at room temperature. Off-white solid appeared, which was filtered to afford 4-[(2,4-dimethoxyphenyl)methyl]-N-(4-nitrophenyl)-3-oxo-2,3,4,5-tetrahydro-1,4-benzoxazepine-5-carboxamide (1.59 g, 49%). LCMS (ES) [M+1]⁺ m/z: 478.2.

### Step 2

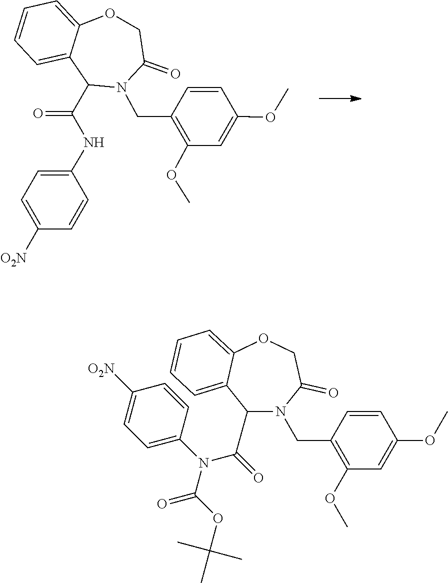 [SMILES: COc1ccc(CN2C(=O)COc3ccccc3C2C(=O)Nc2ccc([N+](=O)[O-])cc2)c(OC)c1 ; COc1ccc(CN2C(=O)COc3ccccc3C2C(=O)N(C(=O)OC(C)(C)C)c2ccc([N+](=O)[O-])cc2)c(OC)c1]

Di-tert-butyl dicarbonate (727 mg, 3.3 mmol, 1.0 eq.) was added to a solution containing 4-[(2,4-dimethoxyphenyl)methyl]-N-(4-nitrophenyl)-3-oxo-2,3,4,5-tetrahydro-1,4-benzoxazepine-5-carboxamide (1.59 g, 3.3 mmol, 1.0 eq.) and N,N-dimethylaminopyridine (81 mg, 0.7 mmol; 0.2 eq.) in THF (17 mL). The reaction mixed was stirred for 1 hour and then diluted with ethyl acetate. The organic layer was washed with saturated ammonium chloride, dried over MgSO₄, filtered, and concentrated to afford crude tert-butyl N-{4-[(2,4-dimethoxyphenyl)methyl]-3-oxo-2,3,4,5-tetrahydro-1,4-benzoxazepine-5-carbonyl}-N-(4-nitrophenyl)carbamate (1.92 g; 99%). LCMS (ES) [M+1]⁺ m/z: 578.2.

### Step 3

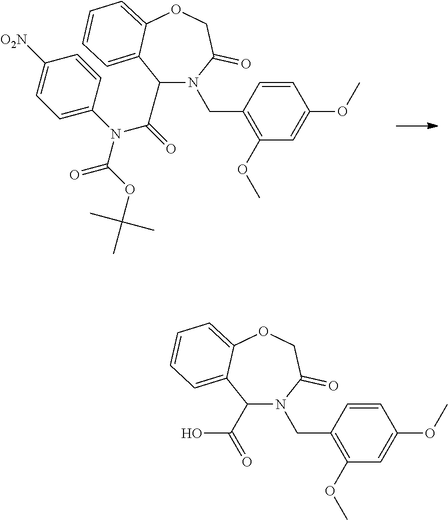 [SMILES: COc1ccc(CN2C(=O)COc3ccccc3C2C(=O)N(C(=O)OC(C)(C)C)c2ccc([N+](=O)[O-])cc2)c(OC)c1 ; COc1ccc(CN2C(=O)COc3ccccc3C2C(=O)O)c(OC)c1]

2M NaOH solution (5.0 mL, 10.0 mmol, 3.0 eq.) was added to a solution of tert-butyl N-{4-[(2,4-dimethoxyphenyl)methyl]-3-oxo-2,3,4,5-tetrahydro-1,4-benzoxazepine-5-carbonyl}-N-(4-nitrophenyl)carbamate (1.92 g, 3.3 mmol, 1.0 eq.) in methanol (17 mL), and the resulting mixture was stirred for 24 hours. The reaction mixture was concentrated under reduced pressure and then partitioned between EtOAc and water. The organic layer was washed with 10% sodium bicarbonate solution. The basic aqueous layers were combined and acidified to pH=3 with 1M HCl. The acidic aqueous layer was extracted with ethyl acetate. The combined organics were dried over MgSO₄, filtered, and concentrated to afford 4-[(2,4-dimethoxyphenyl)methyl]-3-oxo-2,3,4,5-tetrahydro-1,4-benzoxazepine-5-carboxylic acid (1.19 g; 99%). LCMS (ES) [M+1]⁺ m/z: 358.0.

### Step 4

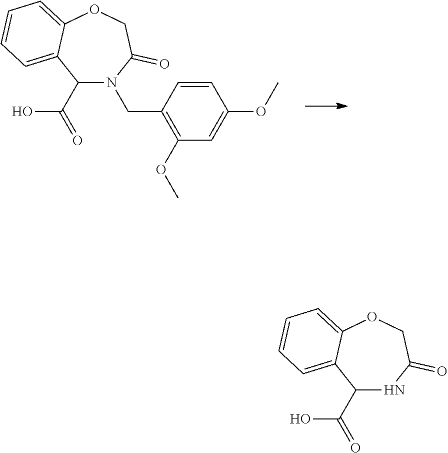 [SMILES: COc1ccc(CN2C(=O)COc3ccccc3C2C(=O)O)c(OC)c1 ; O=C1COc2ccccc2C(C(=O)O)N1]

4-[(2,4-Dimethoxyphenyl)methyl]-3-oxo-2,3,4,5-tetrahydro-1,4-benzoxazepine-5-carboxylic acid (1.19 g, 3.3 mmol, 1.0 eq.) was dissolved in TFA (11 mL) and the reaction mixture was heated to 55° C. for 4 hours. After cooling to room temperature, MeOH (33 mL) was added to the reaction mixture, which caused the precipitation of a purple solid. This solid was removed by filtration, washed with hot DCM/MeOH (10:1), and filtered. The filtrate was concentrated to afford 3-oxo-2,3,4,5-tetrahydro-1,4-benzoxazepine-5-carboxylic acid (437 mg, 63%). LCMS (ES) [M+1]⁺ m/z: 207.9.

### Step 5

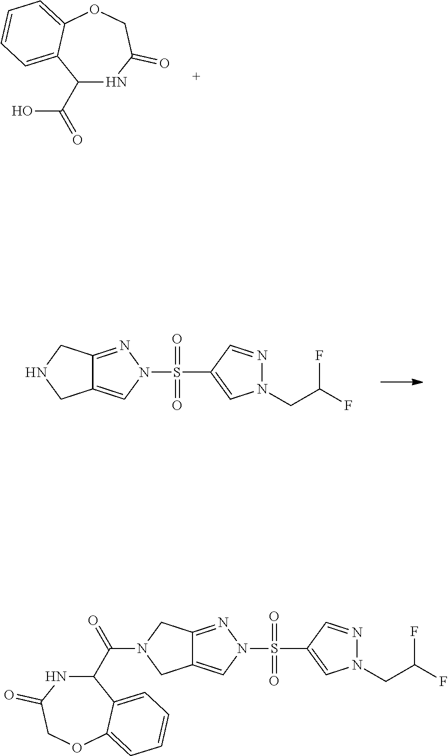 [SMILES: O=C1COc2ccccc2C(C(=O)O)N1 ; O=S(=O)(c1cnn(CC(F)F)c1)n1cc2c(n1)CNC2 ; O=C1COc2ccccc2C(C(=O)N2Cc3cn(S(=O)(=O)c4cnn(CC(F)F)c4)nc3C2)N1]

HATU (184 mg, 0.48 mmol, 1.0 eq.) was added to a solution containing 3-oxo-2,3,4,5-tetrahydro-1,4-benzoxazepine-5-carboxylic acid (100 mg, 0.48 mmol, 1.0 eq.), 1-(2,2-difluoroethyl)-4-{2H,4H,5H,6H-pyrrolo[3,4-c]pyrazole-2-sulfonyl}-1H-pyrazole (Intermediate I-7, 146 mg, 0.48 mmol, 1.0 eq.), triethylamine (0.10 mL; 0.72 mmol; 1.50 eq.), and DMSO (2.4 mL), and the reaction was stirred for 20 minutes. The reaction was then purified by reverse phase prep HPLC (Prep-C18, 5 μM Luna C18 column, 22×250 mm, Phenomenex) eluting with 25% to 60% MeCN in 0.1% FA water to afford 5-(2-{[1-(2,2-difluoroethyl)-1H-pyrazol-4-yl]sulfonyl}-2H,4H,5H,6H-pyrrolo[3,4-c]pyrazole-5-carbonyl)-2,3,4,5-tetrahydro-1,4-benzoxazepin-3-one (66 mg; 28%). ¹HNMR (400 MHz, DMSO-d₆) δ 8.82-8.62 (m, 1H), 8.35-8.00 (m, 2H), 7.51 (dddd, J=15.6, 7.6, 4.0, 1.6 Hz, 1H), 7.34 (tdd, J=7.8, 4.0, 1.6 Hz, 1H), 7.25-7.02 (m, 2H), 6.58-6.15 (m, 1H), 5.41-5.07 (m, 1H), 4.90-4.73 (m, 1H), 4.73-4.61 (m, 2H), 4.60-4.42 (m, 2H), 4.36-4.18 (m, 1H), 3.79 (dd, J=14.1, 10.6 Hz, 1H). LCMS (ES) [M+1]⁺ m/z: 493.1.

### Example 1.9

### Synthesis of 1-(2-{[1-(2,2-difluoroethyl)-1H-pyrazol-4-yl]sulfonyl}-2H,4H,5H,6H-pyrrolo[3,4-c]pyrazole-5-carbonyl)-1,2,3,4-tetrahydroisoquinolin-3-one (Compound 6)

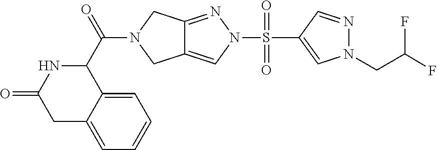 [SMILES: O=C1Cc2ccccc2C(C(=O)N2Cc3cn(S(=O)(=O)c4cnn(CC(F)F)c4)nc3C2)N1]

HATU (199 mg, 0.5 mmol, 1.0 eq.) was added to a solution containing 3-oxo-1,2,3,4-tetrahydroisoquinoline-1-carboxylic acid (100 mg, 0.5 mmol, 1.0 eq.), 1-(2,2-difluoroethyl)-4-{2H,4H,5H,6H-pyrrolo[3,4-c]pyrazole-2-sulfonyl}-1H-pyrazole (Intermediate I-7, 159 mg, 0.52 mmol, 1.0 eq.), triethylamine (0.1 mL, 0.8 mmol, 1.5 eq.), and DMF (2.6 mL). The reaction was allowed to stir for 10 minutes. It was then purified by prep C18 HPLC (Prep-C18, 5 μM Luna C18 column, 22×250 mm, Phenomenex) eluting with 5% to 70% MeCN in 0.1% formic acid in water to afford 1-(2-{[1-(2,2-difluoroethyl)-1H-pyrazol-4-yl]sulfonyl}-2H,4H,5H,6H-pyrrolo[3,4-c]pyrazole-5-carbonyl)-1,2,3,4-tetrahydroisoquinolin-3-one (100 mg, 40%). ¹HNMR (400 MHz, DMSO-d₆) δ 8.75 (ddd, J=27.1, 5.5, 0.7 Hz, 1H), 8.35-7.69 (m, 3H), 7.51-7.33 (m, 1H), 7.30-7.10 (m, 3H), 6.59-6.17 (m, 1H), 5.38 (dq, J=11.0, 6.2, 4.9 Hz, 1H), 5.12-4.87 (m, 2H), 4.81-4.55 (m, 2H), 4.52-4.19 (m, 2H), 3.80 (dd, J=19.3, 13.7 Hz, 1H), 3.34 (dd, J=7.4, 4.1 Hz, 1H). LCMS (ES) [M+1]⁺ m/z: 477.0.

### Example 1.10

### Synthesis of 5-(2-{[1-(2,2-difluoroethyl)-1H-pyrazol-4-yl]sulfonyl}-2H,4H,5H,6H-pyrrolo[3,4-c]pyrazole-5-carbonyl)-9-fluoro-2,3,4,5-tetrahydro-1,4-benzoxazepin-3-one (Compound 7)

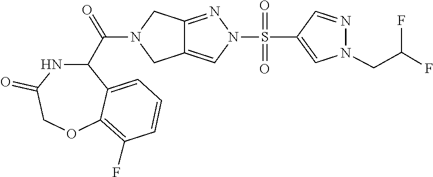 [SMILES: O=C1COc2c(F)cccc2C(C(=O)N2Cc3cn(S(=O)(=O)c4cnn(CC(F)F)c4)nc3C2)N1]

The title compound was made from 2-(2-fluoro-6-formylphenoxy)acetic acid following a 5-step procedure similar to the one described in Example 1.8 for Compound 5. The final crude product was purified by reverse phase preparative HPLC (Prep-C18, 5 μM Luna C18 column, 22×250 mm, Phenomenex) gradient elution of 5-70% MeCN in water containing 0.1% formic acid to provide 5-(2-{[1-(2,2-difluoroethyl)-1H-pyrazol-4-yl]sulfonyl}-2H,4H,5H,6H-pyrrolo[3,4-c]pyrazole-5-carbonyl)-9-fluoro-2,3,4,5-tetrahydro-1,4-benzoxazepin-3-one as a white solid. ¹HNMR (400 MHz, DMSO-d₆) δ 8.85-8.59 (m, 1H), 8.47-8.25 (m, 1H), 8.17-8.06 (m, 2H), 7.45-7.23 (m, 2H), 7.17 (td, J=8.0, 5.0 Hz, 1H), 6.56-6.16 (m, 1H), 5.45-5.19 (m, 1H), 4.94-4.26 (m, 7H), 3.91-3.73 (m, 1H). LCMS (ES) [M+1]⁺ m/z: 511.0.

### Example 1.11

### Synthesis of 3-[2-(2-{[1-(2,2-difluoroethyl)-1H-pyrazol-4-yl]sulfonyl}-2H,4H,5H,6H-pyrrolo[3,4-c]pyrazol-5-yl)-2-oxoethyl]-3-methyl-2,3-dihydro-1H-indol-2-one (Compound 8)

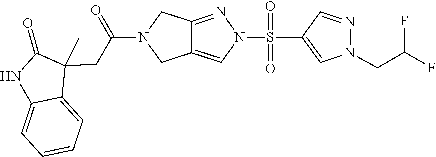 [SMILES: CC1(CC(=O)N2Cc3cn(S(=O)(=O)c4cnn(CC(F)F)c4)nc3C2)C(=O)Nc2ccccc21]

The title compound was synthesized from 1-(2,2-difluoroethyl)-4-{2H,4H,5H,6H-pyrrolo[3,4-c]pyrazole-2-sulfonyl}-1H-pyrazole; trifluoromethanesulfonic acid (Intermediate I-7, 663 mg, 1.5 mol, 1.0 eq.) and 2-(3-methyl-2-oxo-2,3-dihydro-1H-indol-3-yl)acetic acid (300 mg, 1.5 mmol; 1.00 eq.), following the procedure described in Example 1.4 (Compound 1). 3-[2-(2-{[1-(2,2-Difluoroethyl)-1H-pyrazol-4-yl]sulfonyl}-2H,4H,5H,6H-pyrrolo[3,4-c]pyrazol-5-yl)-2-oxoethyl]-3-methyl-2,3-dihydro-1H-indol-2-one (40 mg, 6%) was isolated as a white solid. ¹HNMR (400 MHz, DMSO-d₆) δ 10.16 (d, J=3.4 Hz, 1H), 8.68 (dd, J=7.2, 0.7 Hz, 1H), 8.22-8.07 (m, 2H), 7.23-7.15 (m, 1H), 7.05 (td, J=7.7, 1.3 Hz, 1H), 6.80 (td, J=7.5, 1.0 Hz, 1H), 6.77-6.70 (m, 1H), 6.38 (tdt, J=54.4, 5.3, 3.6 Hz, 1H), 4.79-4.48 (m, 4H), 4.27-4.02 (m, 2H), 3.10 (dd, J=16.9, 2.1 Hz, 1H), 2.84 (dd, J=16.9, 3.4 Hz, 1H), 1.18 (d, J=2.7 Hz, 3H). LCMS (ES) [M+1]⁺ m/z: 491.0.

### Example 1.12

### Synthesis of 3-{2-[2-(1,3-benzothiazole-6-sulfonyl)-2H,4H,5H,6H-pyrrolo[3,4-c]pyrazol-5-yl]-2-oxoethyl}-2,3-dihydro-1H-indol-2-one (Compound 9)

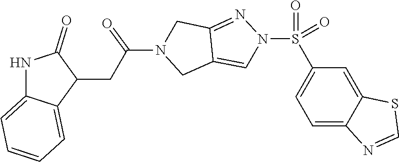

### Step 1

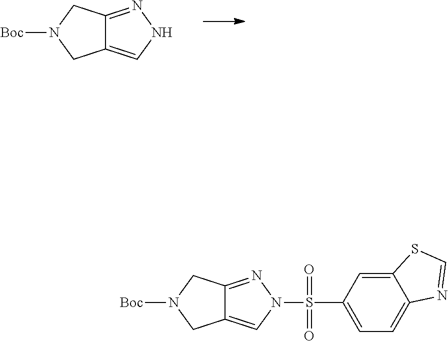 [SMILES: CC(C)(C)OC(=O)N1Cc2c[nH]nc2C1 ; CC(C)(C)OC(=O)N1Cc2cn(S(=O)(=O)c3ccc4ncsc4c3)nc2C1]

To a suspension of sodium hydride (0.70 g; 60%, 17.41 mmol; 1.30 eq.) in dry THF (15 mL) in an ice/water bath under an N₂ atmosphere was added a solution of N-{2H,4H,5H,6H-pyrrolo[3,4-c]pyrazol-5-yl}(tert-butoxy)formamide (3.0 g; 13.40 mmol; 1.00 eq.) in THF (15 mL) over 5 min. The mixture was allowed to stir in the ice bath for 30 min. A solution of 1,3-benzothiazole-6-sulfonyl chloride (3130.39 mg; 13.40 mmol; 1.00 eq.) in THF (15 mL) was added dropwise over 5 min. The mixture was allowed to stir in the ice bath for 45 min, and at rt for 3 h subsequently. To the resulting mixture under N₂ atmosphere was added aq. NH₄Cl solution (15 mL) and HOAc to adjust the pH to ˜5. The organic layer was separated, and the aqueous layer was back extracted with 10% MeOH/CH₂Cl₂ (15 ml) thrice. The combined organic solutions were washed with water and brine. Removal of the volatiles under reduced pressure provided the desired crude product of tert-butyl 2-(1,3-benzothiazole-6-sulfonyl)-2H,4H,5H,6H-pyrrolo[3,4-c]pyrazole-5-carboxylate as a pale yellow solid (5.1 g, 90°/), which was used as-is in the next step. ¹H NMR (DMSO-d₆) δ: 9.70 (s, 1H), 9.02 (s, 1H), 8.37-8.17 (m, 2H), 8.03 (dt, J=8.7, 1.6 Hz, 1H), 4.30 (dd, J=9.8, 3.8 Hz, 4H), 1.39 (s, 9H); LCMS (ES) [M+1]⁺ m/z: 407.3.

### Step 2

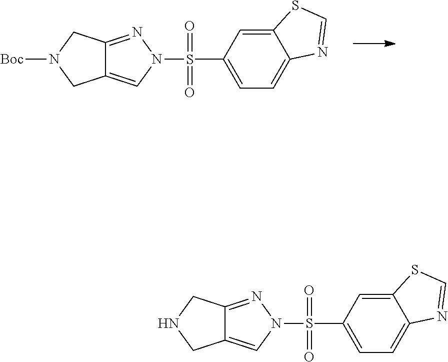 [SMILES: CC(C)(C)OC(=O)N1Cc2cn(S(=O)(=O)c3ccc4ncsc4c3)nc2C1 ; O=S(=O)(c1ccc2ncsc2c1)n1cc2c(n1)CNC2]

To a solution of tert-butyl 2-(1,3-benzothiazole-6-sulfonyl)-2H,4H,5H,6H-pyrrolo[3,4-c]pyrazole-5-carboxylate (4.1 g; 10.0 mol; 1.00 eq.) in CH₂Cl₂ (10 mL) at rt was added a solution of HCl in dioxane (15 mL; 4.00 mol/L; 60 mmol; 6.00 eq.). After stirring at rt for 75 min, the resulting precipitate was filtered, washed with cold CH₂Cl₂ (15 ml) twice, and left on high vacuum overnight to provide the hydrogen chloride salt of the title compound as a white solid (2.86, 93%). ¹H NMR (DMSO-d₆) δ: 10.22 (s, 2H), 9.72 (s, 1H), 9.07 (d, J=2.0 Hz, 1H), 8.45-8.20 (m, 2H), 8.07 (dd, J=8.7, 2.1 Hz, 1H), 4.27 (d, J=7.1 Hz, 4H). LCMS (ES) [M+1]⁺ m/z: 307.3.

### Step 3

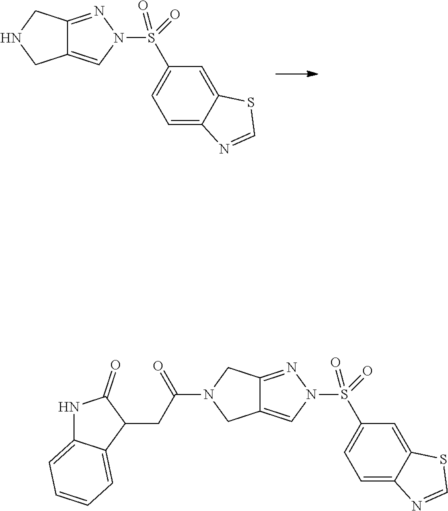 [SMILES: O=S(=O)(c1ccc2ncsc2c1)n1cc2c(n1)CNC2 ; O=C1Nc2ccccc2C1CC(=O)N1Cc2cn(S(=O)(=O)c3ccc4ncsc4c3)nc2C1]

To a solution of 6-{2H,4H,5H,6H-pyrrolo[3,4-c]pyrazole-2-sulfonyl}-1,3-benzothiazole (250.00 mg; 0.82 mmol; 1.00 eq.) and (2-oxo-2,3-dihydro-1H-indol-3-yl)acetic acid (156.01 mg; 0.82 mmol; 1.00 eq.) in DMF (8.75 mL) was added diisopropylethylamine (0.43 mL; 2.45 mmol; 3.00 eq.) followed by HATU (310.28 mg; 0.82 mmol; 1.00 eq.) and the reaction mix was left stirring at RT overnight. The reaction mixture was cooled to 0° C. and treated drop-wise with the addition of water. A precipitate formed that was removed by filtration. The crude solid was taken in DMSO and purified by reverse phase preparative HPLC (Prep-C18, 5 μM XBridge column, 19×150 mm, Waters; gradient elution of 10-80% MeCN in water over a 20 min period, where both solvents contained 0.1% formic acid) to provide the title product as a white solid (51 mg, 13%). ¹H NMR (400 MHz, DMSO-d6) δ 10.30 (d, J=2.5 Hz, 1H), 9.68 (s, 1H), 9.00 (d, J=2.0 Hz, 1H), 8.37-8.20 (m, 2H), 8.02 (ddd, J=8.7, 2.0, 0.9 Hz, 1H), 7.20-7.00 (m, 2H), 6.87-6.67 (m, 2H), 4.69-4.47 (m, 2H), 4.43-4.18 (m, 2H), 3.66 (dt, J=7.7, 3.6 Hz, 1H), 3.01 (dt, J=17.0, 3.8 Hz, 1H), 2.73 (ddd, J=17.1, 8.2, 6.1 Hz, 1H). LCMS (ES) [M+1]⁺ m/z: 480.65.

### Example 1.13

### Synthesis of 3-(2-{2-[4-(difluoromethoxy)benzenesulfonyl]-2H,4H,5H,6H-pyrrolo[3,4-c]pyrazol-5-yl}-2-oxoethyl)-2,3-dihydro-1H-indol-2-one (Compound 10)

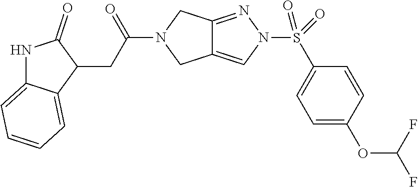 [SMILES: O=C1Nc2ccccc2C1CC(=O)N1Cc2cn(S(=O)(=O)c3ccc(OC(F)F)cc3)nc2C1]

### Step 1

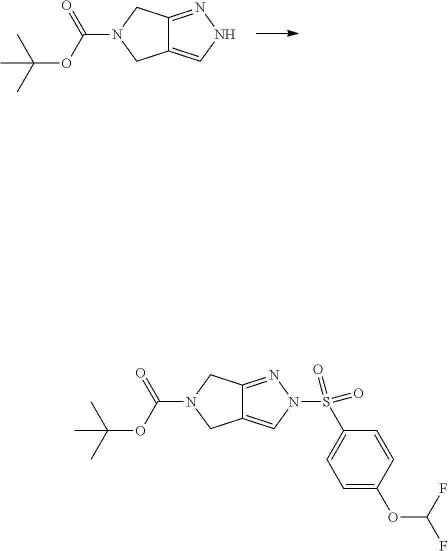 [SMILES: CC(C)(C)OC(=O)N1Cc2c[nH]nc2C1 ; CC(C)(C)OC(=O)N1Cc2cn(S(=O)(=O)c3ccc(OC(F)F)cc3)nc2C1]

To a suspension of sodium hydride (1.24 g; 31.06 mmol; 1.30 eq.) in THF (200.00 mL) under N₂ in ice bath was added a solution of tert-butyl 2H,4H,5H,6H-pyrrolo[3,4-c]pyrazole-5-carboxylate (5.00 g; 23.90 mmol; 1.00 eq.) in THF (70 mL) over 10 min. The mixture was allowed to stir in the ice bath for 30 min. A solution of 4-(difluoromethoxy)benzenesulfonyl chloride (6.38 g; 26.28 mmol; 1.10 eq.) in THF (30 mL) was then added slowly. The mixture was allowed to stir in the ice bath for 45 min and then allowed to warm to rt. After 90 min at RT the reaction mixture was quenched and diluted with NH₄Cl (5 mL of AcOH was also added). This mixture was extracted twice with a 1:3 mixture of IPA:CHCl₃. The combined organics were washed with water and brine, dried over Na₂SO₄, filtered, and concentrated to provide an orange oil, which solidified overnight. The crude material was purified on a silica gel column using 0-30% EtOAc in heptanes to give tert-butyl 2-[4-(difluoromethoxy)benzenesulfonyl]-2H,4H,5H,6H-pyrrolo[3,4-c]pyrazole-5-carboxylate (9 g, 90.8%). LCMS (ES) [M+1]⁺ m/z: 416.2.

### Step 2

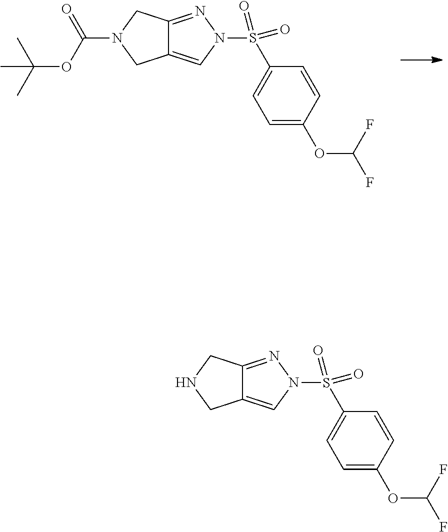 [SMILES: CC(C)(C)OC(=O)N1Cc2cn(S(=O)(=O)c3ccc(OC(F)F)cc3)nc2C1 ; O=S(=O)(c1ccc(OC(F)F)cc1)n1cc2c(n1)CNC2]

To a solution of tert-butyl 2-[4-(difluoromethoxy)benzenesulfonyl]-2H,4H,5H,6H-pyrrolo[3,4-c]pyrazole-5-carboxylate (9.00 g; 21.67 mmol; 1.00 eq.) in 1,2-dichloroethane (540.00 mL) was added ZnBr₂ (14.64 g; 65.00 mmol; 3.00 eq.). This mixture was allowed to stir at 60° C. overnight. The reaction was cooled to RT, diluted with water, and treated with 3 ml of concentrated ammonium hydroxide. The compound was extracted three times with a 3:1 mixture of CHCl₃:IPA, washed with brine, and dried over MgSO₄. After removal of the volatiles under reduced pressure, the crude 2-[4-(difluoromethoxy)benzenesulfonyl]-2H,4H,5H,6H-pyrrolo[3,4-c]pyrazole was used as-is in the next step. LCMS (ES) [M+1]⁺ m/z: 316.

### Step 3

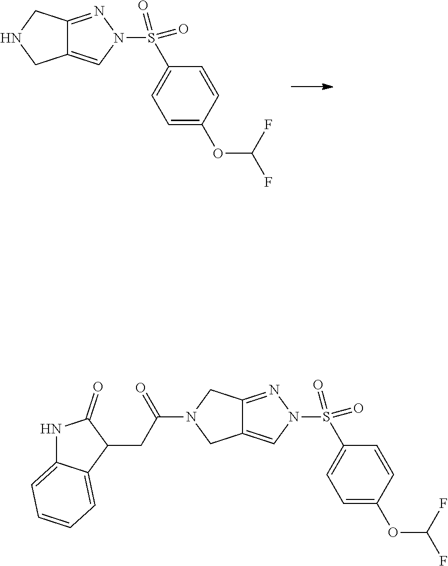 [SMILES: O=S(=O)(c1ccc(OC(F)F)cc1)n1cc2c(n1)CNC2 ; O=C1Nc2ccccc2C1CC(=O)N1Cc2cn(S(=O)(=O)c3ccc(OC(F)F)cc3)nc2C1]

To a solution of 2-[4-(difluoromethoxy)benzenesulfonyl]-2H,4H,5H,6H-pyrrolo[3,4-c]pyrazole (250.00 mg; 0.79 mmol; 1.00 eq.) and (2-oxo-2,3-dihydro-1H-indol-3-yl)acetic acid (151.59 mg; 0.79 mmol; 1.00 eq.) in DMF (8.75 mL) were added Hunig's base (0.41 mL; 2.38 mmol; 3.00 eq.) and HATU (301.49 mg; 0.79 mmol; 1.00 eq.) and the reaction mix was left stirring at RT overnight. The reaction mix was cooled to 0° C. and treated with drop-wise with the addition of water. A precipitate formed that was removed by filtration. The crude solid was subjected to purification on a reverse phase silica gel column using 0-50% acetonitrile in water to give 3-(2-{2-[4-(difluoromethoxy)benzenesulfonyl]-2H,4H,5H,6H-pyrrolo[3,4-c]pyrazol-5-yl}-2-oxoethyl)-2,3-dihydro-1H-indol-2-one as a white solid (170 mg, 43.9%). ¹H NMR (400 MHz, DMSO-d6) δ 10.31 (s, 1H), 8.32-8.21 (m, 1H), 8.08-7.95 (m, 2H), 7.61-7.20 (m, 3H), 7.19-7.13 (m, 1H), 7.11-7.05 (m, 1H), 6.82 (td, J=7.5, 1.1 Hz, 1H), 6.76 (d, J=7.7 Hz, 1H), 4.60 (dd, J=17.7, 5.0 Hz, 2H), 4.43-4.27 (m, 2H), 3.67 (d, J=7.8 Hz, 1H), 3.03 (dd, J=17.0, 3.7 Hz, 1H), 2.75 (ddd, J=17.0, 8.2, 2.8 Hz, 1H). LCMS (ES) [M+1]⁺ m/z: 489.8.

### Example 1.14

### Synthesis of 1-(2-{2-[4-(difluoromethoxy)benzenesulfonyl]-2H,4H,5H,6H-pyrrolo[3,4-c]pyrazol-5-yl}-2-oxoethyl)-2,3-dihydro-1H-indol-2-one (Compound 11)

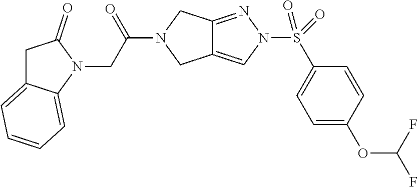 [SMILES: O=C(CN1C(=O)Cc2ccccc21)N1Cc2cn(S(=O)(=O)c3ccc(OC(F)F)cc3)nc2C1]

The title compound was synthesized according to the procedure described in Step 3, Example 13 (Compound 10) using 2-[4-(difluoromethoxy)benzenesulfonyl]-2H,4H,5H,6H-pyrrolo[3,4-c]pyrazole (120.00 mg; 0.38 mmol; 1.00 eq.) and 2-(2-oxo-2,3-dihydro-1H-indol-1-yl)acetic acid (72.76 mg; 0.38 mmol; 1.00 eq.). 1-(2-{2-[4-(difluoromethoxy)benzenesulfonyl]-2H,4H,5H,6H-pyrrolo[3,4-c]pyrazol-5-yl}-2-oxoethyl)-2,3-dihydro-1H-indol-2-one was isolated as a white solid (185.9 mg, 82%). ¹H NMR (400 MHz, DMSO-d₆) δ 8.43-8.23 (m, 1H), 8.07-8.00 (m, 2H), 7.47-7.36 (m, 3H), 7.25-7.20 (m, 1H), 7.16 (t, J=7.6 Hz, 1H), 6.95 (td, J=7.7, 1.1 Hz, 1H), 6.89-6.82 (m, 1H), 4.80 (d, J=15.8 Hz, 2H), 4.56 (d, J=7.3 Hz, 2H), 4.43-4.32 (m, 2H), 3.58 (s, 2H). LCMS (ES) [M+1]⁺ m/z: 489.3.

### Example 1.15

### Synthesis of 5-{2-[4-(difluoromethoxy)benzenesulfonyl]-2H,4H,5H,6H-pyrrolo[3,4-c]pyrazole-5-carbonyl}-2,3,4,5-tetrahydro-1,4-benzoxazepin-3-one (Compound 12)

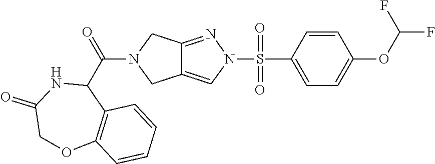 [SMILES: O=C1COc2ccccc2C(C(=O)N2Cc3cn(S(=O)(=O)c4ccc(OC(F)F)cc4)nc3C2)N1]

HATU (183 mg, 0.45 mmol, 1.0 eq.) was added to a solution containing 2-[4-(difluoromethoxy)benzenesulfonyl]-2H,4H,5H,6H-pyrrolo[3,4-c]pyrazole (152 mg, 0.45 mmol, 1.0 eq.), 3-oxo-2,3,4,5-tetrahydro-1,4-benzoxazepine-5-carboxylic acid (100 mg, 0.45 mmol, 1.0 eq.), and triethylamine (0.10 mL, 0.68 mmol, 1.50 eq.) in DMSO (3 mL), and the reaction was stirred for 15 minutes. The crude was purified by prep C18 HPLC (Prep-C18, 5 μM Luna C₁₈ column, 22×250 mm, Phenomenex) eluting with 5% to 70% MeCN in 0.1% formic acid in water to afford 5-{2-[4-(difluoromethoxy)benzenesulfonyl]-2H,4H,5H,6H-pyrrolo[3,4-c]pyrazole-5-carbonyl}-2,3,4,5-tetrahydro-1,4-benzoxazepin-3-one (53 mg, 22%). ¹H NMR (400 MHz, DMSO-d₆) δ 8.31-8.13 (m, 2H), 8.02-7.94 (m, 2H), 7.50 (ddd, J=16.9, 7.6, 1.7 Hz, 1H), 7.41-7.35 (m, 3H), 7.35-7.29 (m, 1H), 7.16 (tt, J=7.5, 1.2 Hz, 1H), 7.07 (ddd, J=9.3, 8.0, 1.2 Hz, 1H), 5.25 (dd, J=47.2, 6.3 Hz, 1H), 4.79 (dd, J=29.3, 14.2 Hz, 1H), 4.59-4.17 (m, 5H), 3.76 (dd, J=15.8, 14.3 Hz, 1H). LCMS (ES) [M+1]⁺ m/z: 505.1.

### II. Biological Examples

### Example 2.1

### Pyruvate Kinase Activation Methods

### Protein Production and Purification

Human pyruvate kinase R (PKR), residues 14-574, and human pyruvate kinase M2, residues 1-531, were cloned into expression plasmids and obtained from ATUM Bio (Newark, CA). Proteins were translated as fusions with 6×-His,8×-Arg, and SUMO at the N-terminus. A third construct of human PKR was truncated, residues 50-574, and similarly cloned for use in crystallography experiments. All proteins were cloned, expressed in E. coli, and purified using similar protocols. Cells were grown at 30° C. in Luria-Bertani broth supplemented with 0.4% glucose to OD600=0.8 and induced with 0.4 mM IPTG at 16° C. for 18 hours. Cells were harvested by centrifugation, resuspended in 50 mM potassium phosphate pH 8.0, 500 mM NaCl, 25 mM imidazole pH 8.0, and 3 mM β-mercaptoethanol. Cells were lysed using a LM20 microfluidizer from Microfluidics (Westwood, MA). The crude lysate was immediately supplemented with 0.2 mM phenylmethylsulfonyl fluoride (PMSF) and centrifuged at 20,000 g for 20 minutes. The soluble fraction was subsequently incubated with 2 mL Ni-NTA (GE Healthcare) per 1,000 ODs for 1 hour at 4° C. Following incubation with the Ni-NTA resin, lysate was removed by pelleting resin at 2,500 g for 3 minutes and washed 3 times with 9 bed volumes of 50 mM potassium phosphate pH 8.0, 500 mM NaCl, 25 mM imidazole, and 3 mM β-mercaptoethanol. Following the batch wash Ni-NTA resin was loaded onto a gravity column and His-tagged protein was eluted with 6 bed volumes of 10 mM Tris/HCl pH 8.0, 200 mM NaCl, 500 mM imidazole, and 3 mM β-mercaptoethanol. Eluted protein was dialyzed overnight against 10 mM Tris/HCl pH 8.0, 200 mM NaCl, and 1 mM DTT and the 6×-His-8×-Arg-SUMO-tag was cleaved using a 20:1 molar ratio of protein:3C protease. The protein was purified by anion exchange chromatography on a HiTrapQ or MonoQ 10/100 GL column (GE Healthcare) via a linear NaCl gradient and twice by size exclusion chromatography using a Superdex S200 26/60 column (GE Healthcare) run in 10 mM Tris/HCl pH 8.0, 200 mM NaCl, 10 mM MgCl₂, 1 mM DTT. Proteins were concentrated to ˜12 mg/mL for crystallization and flash frozen for storage.

### Example 2.2

### Biochemical Assay for Activation of Pyruvate Kinase R

Activation of pyruvate kinase R (PKR) and pyruvate kinase M2 (PKM2) were measured using a luminescence based measurement of ATP generation and the Kinase-Glo luminescent kinase reagent kit. 0.1 nM of PKR or PKM2 was incubated for 1 hour with 0.3 mM ADP, 2 mM FBP, 1% DMSO, compound and Assay Buffer mix (500 mM Tris/HCl pH 7.5, 500 mM NaCl, 50 mM MgCl₂, BSA, and 2 mM DTT, in a total reaction volume of 30 uL. After one hour PEP was added to the reaction at a final concentration of 0.1 mM and incubated for another hour at room temperature. 30 uL of KinaseGlo reagent was added and the reaction was incubated for 15 minutes. Endpoint luminescence data was measured using an EnVision plate reader (PerkinElmer). The assay results are provided in Table 2 for PKR.

TABLE 2

| Cmpd No. | PKR^{a} | PKR | PKM2^{b} | PKM2 |
|---|---|---|---|---|
| from | AC₅₀ | % | AC₅₀ | % |
| Table 1 | (nM) | activation^{c} | (nM) | activation^{c} |
| 1 | 230 | 212 | | |
| 2 | 410 | 32 | | |
| 3 | 1800 | 60 | | |
| 4 | 10,000 | 78 | | |
| 5 | 490 | 91 | | |
| 6 | 4400 | 84 | | |
| 7 | 1800 | 66 | | |
| 8 | 1900 | 95 | | |
| 9 | 28 | 161 | 100 | 87 |
| 10 | 78 | 129 | 14 | 165 |
| 11 | 1700 | 77 | | |
| 12 | 210 | 91 | | |

^{a}Activity data was measured in PKR biochemical assay.
^{b}Activity data was measured in PKM2 cellular assay.
^{c}The maximal activation level achieved with each compound relative to the activation level achieved by the literature compound AG-348 @ 10 μM.

### Example 2.3

### Cell Based Assay for Activation of Pyruvate Kinase M2 in Lung Carcinoma Cell Line

H1299 cells were seeded in 96-well plates at 2,000 cells/well (100 uL). Treated plates were incubated overnight at 37° C. with 5% CO₂. Compounds were diluted in complete media and added to cells in the presence of 1% DMSO. Cells were incubated with compound for 90 minutes at 37° C. and 5% CO₂ before washing three times with PBS to remove residual compound and then lysed in lysis buffer (Cell Signaling). Cell lysate was analyzed using the NADH coupled assay described below with a reaction mixture of 180 uM NADH, 2 mM ADP, 0.5 units of LDH and 0.5 mM of PEP. PKM2 activity was measured at steady state using a coupled enzyme activation system based on NADH consumption. PKM2 produces pyruvate and the coupled system uses lactate dehydrogenase (LDH) to reduce pyruvate to lactate with the concomitant oxidation of NADH to NAD⁺. Conversion of NADH to NAD⁺ was monitored using a SPECTROstar Nano plate reader (BMG Labtech) at a wavelength of 340 nm and subtraction of background absorbance measured at 750 nm. The change in NADH absorbance after PEP addition was monitored and slope obtained by subtracting baseline at 750 nm followed by least squares fitting to a simple linear regression model. A 10-point curve was generated to calculate the AC₅₀ values by fitting the rates of NADH consumption against increasing concentration of compound. The assay results are provided in Table 2.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A compound of Formula I: 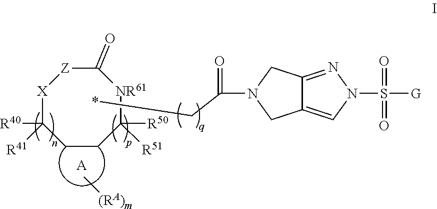
or a pharmaceutically acceptable salt thereof;
wherein,
m is 0, 1, or 2;
Ring A is a fused phenyl or 5- or 6-membered heteroaryl;
R^{A}, in each instance, is independently selected from the group consisting of C₁-C₃ alkyl, C₁-C₃ alkoxy, hydroxy, halo-C₁-C₃ alkyl, halo-C₁-C₃ alkoxy, and halo;
Z is a bond or CR²⁰R²¹;
wherein, R²⁰ and R²¹ are each independently hydrogen or C₁-C₃ alkyl;
X is NR³⁰, O or CR³⁰R³¹;
wherein, R³⁰ and R³¹ are each independently hydrogen or C₁-C₃ alkyl;
R⁴⁰, R⁴¹, R⁵⁰, R⁵¹ and R⁶¹ are each independently hydrogen or C₁-C₃ alkyl;
n is 0 or 1;
p is 0 or 1;
* is an attachment point at any one of R²⁰, R²¹, R³⁰, R³¹, R⁴⁰, R⁴¹, R⁵⁰, R⁵¹ and R⁶¹;
q is 0 or 1;
G is:
a) a 5-membered heteroaryl ring optionally substituted with one or two substituents, each independently selected from the group consisting of halo, C₁-C₃ alkyl, C₁-C₃ alkoxy, halo-C₁-C₃ alkoxy, and halo-C₁-C₃ alkyl;
or,
b) 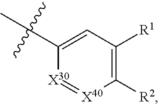
wherein,
X³⁰ and X⁴⁰ are each independently N or CH, provided that only one of X³⁰ and X⁴⁰ can be N;
R¹ and R² are each independently selected from the group consisting of hydrogen, C₁-C₃ alkyl, amino, halo, halo-C₁-C₃ alkyl, halo-C₁-C₃ alkoxy, C₁-C₃ alkoxy, hydroxy, —O—R^{aa}, —(C₁-C₃ alkoxy)-R^{aa}, and C₃-C₇ cycloalkyl;
R^{aa} is 4- to 7-membered heterocyclyl or C₃-C₇ cycloalkyl;
or,
R¹ and R², together with the carbon to which each is attached, form a 5- or 6-membered heteroaryl or 5- to 7-membered heterocyclyl containing one or two heteroatoms;
wherein said heteroaryl or heterocyclyl comprising R¹ and R² is optionally substituted with one or two substituents, each independently selected from the group consisting of C₁-C₃ alkyl, halo-C₁-C₃ alkyl, C₁-C₃ alkoxy, halo, and halo-C₁-C₃ alkoxy.

2. The compound of claim 1, wherein ring A is phenyl and the compound is of Formula Ia:

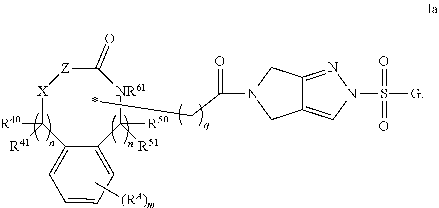

3. The compound of claim 1 or 2, wherein Z is a bond.

4. The compound of claim 1 or 2, wherein Z is CR²⁰R²¹.

5. The compound of claim 4, wherein R²⁰ and R²¹ are each hydrogen.

6. The compound of any one of claims 1-5, wherein X is CR³⁰R³¹.

7. The compound of claim 6, wherein R³⁰ is hydrogen.

8. The compound of claim 6, wherein R³⁰ is methyl.

9. The compound of claim 6, wherein R³¹ is * and the compound is of Formula Ib:

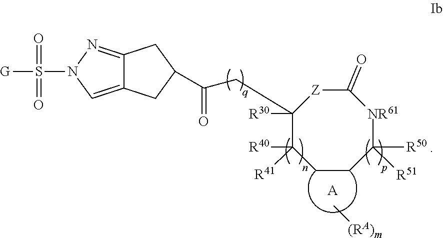

10. The compound of claim 6, wherein R³¹ is hydrogen.

11. The compound of any one of claims 1-5, wherein X is O.

12. The compound of any one of claims 1-5, wherein X is NR³⁰.

13. The compound of claim 12, wherein R³⁰ is hydrogen.

14. The compound of any one of claims 1-13, wherein R⁶¹ is hydrogen.

15. The compound of any one of claims 1-8 or 10-13, wherein R⁶¹ is * and the compound is of Formula Ic:

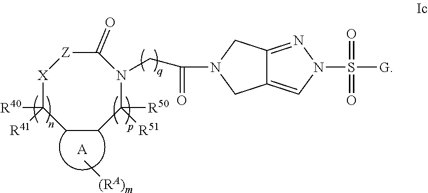

16. The compound of any one of claims 1-15, wherein n is 0.

17. The compound of any one of claims 1-8, 10-14, or 16, wherein p is 1, R⁵⁰ is hydrogen and R⁵¹ is * and the compound is of Formula Id:

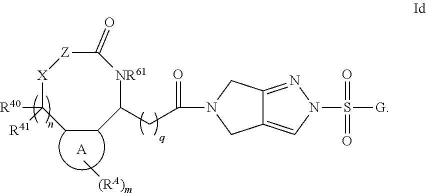

18. The compound of any one of claims 1-16, wherein p is 0.

19. The compound of claim 1 or 2, wherein R³¹ is * and the compound is of Formula Ib: 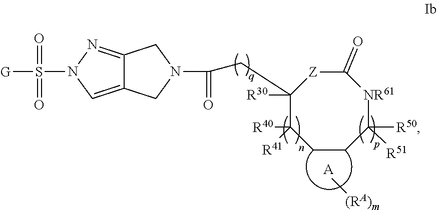
wherein,
Z is a bond;
R³⁰ is hydrogen or methyl;
R⁶¹ is hydrogen;
n is 0; and
p is 0.

20. The compound of claim 1 or 2, wherein R⁶¹ is * and the compound is of Formula IC: 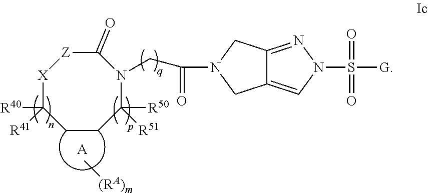
wherein,
Z is a bond;
X is O, NH, or CH₂;
n is 0;
and p is 0.

21. The compound of claim 1 or 2, wherein p is 1, R⁵⁰ is hydrogen, R⁵¹ is * and the compound is of Formula Id: 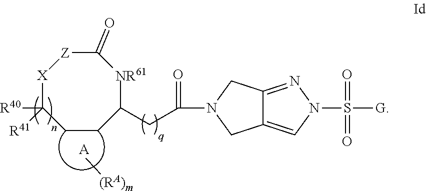
wherein,
Z is CH₂;
X is O;
n is 0; and
R⁶¹ is hydrogen.

22. The compound of claim 1 or 2, wherein p is 1, R⁵⁰ is hydrogen, R⁵¹ is * and the compound is of Formula Id: 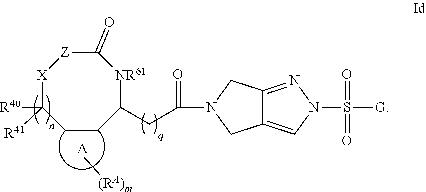
wherein,
Z is a bond;
X is CH₂;
n is 0; and,
R⁶¹ is hydrogen.

23. The compound of any one of claims 1-22, wherein q is 0.

24. The compound of any one of claims 1-22, wherein q is 1.

25. The compound of any one of claims 1-24, wherein G is phenyl, wherein R¹ is hydrogen and R² is halo-C₁-C₃ alkoxy.

26. The compound of claim 25, wherein R² is —OCH₂F, —OCHF₂, or —OCF₃.

27. The compound of claim 26, wherein R² is —OCHF₂.

28. The compound of any one of claims 1-24, wherein G is phenyl, wherein R¹ and R², together with the carbon to which each is attached, form a 5-membered heteroaryl.

29. The compound of claim 28, wherein R¹ and R², together with the carbon to which each is attached, form a thiazolyl.

30. The compound of claim 28 or 29, wherein G is:

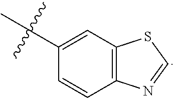 [SMILES: *c1ccc2ncsc2c1]

31. The compound of any one of claims 1-24, wherein G is a 5-membered heteroaryl ring optionally substituted with one or two substituents, each independently selected from the group consisting of halo, halo-C₁-C₃ alkoxy, and halo-C₁-C₃ alkyl.

32. The compound of claim 31, wherein G is a pyrazolyl substituted once with halo-C₁-C₃ alkyl.

33. The compound of claim 32, wherein G is: 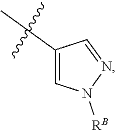
wherein,
R^{B} is selected from the group consisting of —CH₂CH₂F, —CH₂CHF₂, and —CH₂CF₃.

34. The compound of claim 33, wherein R^{B} is —CH₂CHF₂.

35. The compound of any one of claims 1-34, wherein R^{A} is halo.

36. The compound of any one of claims 1-35, wherein m is 0.

37. The compound of any one of claims 1-35, wherein m is 1.

38. The compound of claim 1, wherein the compound is selected from Table 1, or a pharmaceutically acceptable salt thereof.

39. A pharmaceutical composition comprising a compound of any one of claims 1-38 or a pharmaceutically acceptable salt thereof, and a pharmaceutically acceptable excipient.

40. A method of treating a disease or disorder associated with modulation of pyruvate kinase (PKR) and/or PKM2 in a subject, comprising administering to the subject an effective amount of a compound of any one of claims 1-38 or the pharmaceutical composition of claim 39.

41. A method of activating PKR and/or PKM2 in a subject, comprising administering to the subject an effective amount of a compound of any one of claims 1-38 or the pharmaceutical composition of claim 39.

42. A method of treating a subject afflicted with a disease associated with decreased activity of PKR and/or PKM2, comprising administering to the subject an effective amount of a compound of any one of claims 1-38 or the pharmaceutical composition of claim 39.

43. The method of claim 42, wherein the disease is selected from the group consisting of sickle cell disease, sickle cell anemia, thalassemia, hereditary non-spherocytic hemolytic anemia, hemolytic anemia, hereditary spherocytosis, hereditary elliptocytosis, abetalipoproteinemia, paroxysmal nocturnal hemoglobinuria, acquired hemolytic, and anemia of chronic diseases.
